(19) United States
(12) Patent Application Publication
(10) Pub. No.: US 2004/0191769 A1
(43) Pub. Date: Sep. 30, 2004

(54) Methods, compositions, and kits for mutation detection in mitochondrial DNA

(75) Inventors: Michael A. Marino, Frederick, MD (US); Patricia McAndrew, Montgomery Village, MD (US)

Correspondence Address: Keith Johnson, Esq., Transgenomic, Inc., 12325 Emmett Street, Omaha, NE, 68164, US

(73) Assignee: Transgenomic, Inc., San Jose, CA (US)

(21) Appl. No.: 10/202,162

(22) Filed: Jul. 24, 2002

Related U.S. Application Data
(60) Provisional application No. 60/392,911, filed on Jun. 28, 2002.
(60) Provisional application No. 60/307,645, filed on Jul. 24, 2001.

(51) Int. Cl. C12Q 1/68; C12P 19/34

(52) U.S. Cl. 435/6; 435/91.2

(57) ABSTRACT

Methods, compositions, and kits for detecting mutations in the entire human mitochondrial genome. A preferred method includes amplifying mtDNA from a biological sample by polymerase chain reaction of total DNA using a plurality of pre-selected primer pairs to generate overlapping amplicons; cleaving the amplicons using restriction enzymes to produce fragments suitable for analysis by denaturing high performance liquid chromatography (DHPLC); denaturing and re-annealing the amplicons; and fragment analysis by DHPLC to detect the presence or absence of heteroduplex molecules.

### CROSS-REFERENCE TO RELATED PATENT APPLICATIONS

This application is a regular U.S. patent application under 35 U.S.C. §111(a) and 37 U.S.C. §1.53(b) and claims priority from the following co-pending, commonly assigned provisional applications, each filed under 35 U.S.C. §111(b):

U.S. Patent Application No. 60/307,645 filed on Jul. 24, 2001, and U.S. Patent Application No. 60/392,911 and filed on Jun. 28, 2002.

### FIELD OF THE INVENTION

The present invention concerns improved methods, compositions, and kits for detection of mutations in mitochondrial DNA.

### BACKGROUND OF THE INVENTION

Mitochondria are DNA-containing organelles found within the cytoplasm of eukaryotic cells. Their main function is to provide energy for cellular activity in the form of ATP through the process of oxidative phosphorylation. Sequence analysis of the mitochondrial genome (Anderson, S. et al. Nature 290, 457-465 (1981)) revealed this 16.5 kb circular molecule encodes 37 genes necessary for mitochondrial function. Mitochondrial DNA (mtDNA) molecules are maternally inherited and can be present at up to 10,000 copies per cell. It is estimated that the mutation rate in mtDNA is 10× higher than nuclear DNA due to the combined effects of exposure to reactive oxygen species, the lack of protective histones, and the lack of efficient repair mechanisms (Lightowlers, R. N., et al. Trends in Genet. 13, 450-455 (1997); Wallace, D. C., Science, 283, 1482-1488 (1999)).

Molecular analysis of the mitochondrial genome has applications in a wide range of study areas. Defects in the human mitochondrial genome have been reported for several degenerative diseases, cancer, and aging (Fliss, M. S. et al. Science 287, 2017-2019 (2000); Lightowlers, R. N. et al. (1997); Lin, M. T. et al. Hum Mol Genet 11, 133-45 (2002); Polyak, K. et al. Nat Genet 20, 291-293 (1998); Yeh, J. J. Oncogene 19, 2060-6066 (1999); Wallace, D. C. (1999)). Mitochondrial DNA analysis is increasingly being utilized in forensic cases where only limited amounts of degraded material are available (Salas, A. (2001)). In addition, the study of mitochondrial DNA has also provided insights into human evolution and genetic population studies (Ingman M, Gyllensten U. J Hered, 92, 454-61 (2001)).

There is a need for a rapid, sensitive method to screen the human mitochondrial genome for base changes. An efficient method to rapidly screen mtDNA for base changes requires an extremely sensitive system capable of detecting low levels of heteroplasmy. This is the coexistence of normal and mutant mtDNA molecules within an individual and can be maternally inherited or arise through somatic mutation. For many degenerative diseases, the proportion and distribution of this heteroplasmy often determines the severity of clinical symptoms (Lightowlers, R. N. et al. (1997); Wallace, D C. (1999). An efficient assay is required because disorders resulting from mtDNA mutations often present with a wide range of clinical symptoms and have complex inheritance patterns. The recent identification of mutations in nuclear genes that cause disease by introducing mutation in mtDNA further complicates the molecular diagnosis of mitochondrial disorders (Ponamarev, M. G. et al. J Biol Chem. 277, 15225-15228 (2002); Spelbrink, J. N., Li F Y, Tiranti V, Nikali K, Yuan Q P, Tariq M, Wanrooij S, Garrido N, Comi G, Morandi L, Santoro L, Toscano A, Fabrizi G M, Somer H, Croxen R, Beeson D, Poulton J, Suomalainen A, Jacobs H T, Zeviani M, Larsson C. (2001) Nat Genet 28, 223-231 (2001)).

### SUMMARY OF THE INVENTION

In one aspect, the invention concerns a method for detecting the presence of one or more mutations in the entire human mitochondrial genome, wherein the genome is present in a biological sample. In a preferred embodiment, the method comprises

(a) amplifying DNA from the biological sample by polymerase chain reaction using a plurality of pre-selected primer pairs, wherein separate amplicons are generated from each primer pair, wherein the primer pairs are selected such that the amplicons comprise overlapping segments of the entire mitochondrial genome;

(b) cleaving at least one of the separate amplicons using pre-selected restriction enzymes, wherein the restriciton enzymes are selected such that, for each of the separate amplicons, the DNA products obtained after the amplifying and the cleaving are suitable for analysis by DHPLC, and are preferably in the size range of between about 50 base pairs and about 700 base pairs in length;

(c) for each of the separate amplicons, denaturing and re-annealing the separate amplicons of step (b);

(d) for each of the separate amplicons, analyzing the product of step (c) using denaturing high performance liquid chromatography, wherein the presence of one or more mutations is confirmed if at least one heteroduplex is detected.

Preferably, DNA is extracted from the biological sample prior to step (a). The method preferably includes confirming that the cleaving in step (b) is complete. This confirmation can include analyzing the product of step (b) by ion-pairing reverse-phase high performance liquid chromatography under non-denaturing conditions, or by using other methods, such as gel eletrophoresis, or capillary electrophoresis. The analysis in step (d) includes applying the product of step (c) to a stationary reverse phase support; and, eluting with a mobile phase containing an ion-paring reagent and an organic solvent, wherein the eluting is carried out under conditions effective to at least partially denature heteroduplex molecules, and wherein the eluting results in the separation, or partial separation, of heteroduplex and homoduplex molecules.

The restriction enzymes can be selected from the group consisting of at least one of AluI, DdeI, HaeIII, MboI, MspI, BfaI, NlaIII, HpaII, TaqI, HinfI, HphI, SfaNI, DpnII, and mixtures thereof. Preferably the enzymes are selected from the group consisting of at least one of MboI, HaeIII, DdeI, MspI, and AluI, and mixtures thereof. Preferably, the restriction enzymes each require about the same reaction temperature for optimal activity. In various embodiments of the inventive method, the number of separate amplicons is at least 5, preferably at least 10, and more preferably at least 20. Also in various embodiments, the number of separate amplicons is in the range of about 1 to about 70, preferably in the range of about 10 to about 50, and more preferably in the range of about 15 to about 25.

Also, in the preferred method, each of the separate amplicons overlaps its adjacent neighboring amplicons, wherein the length of the overlap is at least 50 base pairs.

The length of the overlap is at least 50 base pairs, preferably 100 base pairs, more preferably in the range of about 50 to about 1000 base pairs, and most preferably in the range of about 60 to about 500 base pairs.

The fragments obtained by restriction cleavage of the separate amplions can differ in size by at least 20 base pairs, preferably at least 40 base pairs, more preferably by at least 100 base pairs, and most preferably by at least 300 base pairs.

The biological sample can be obtained from human or mammalian tissue. The sample can include tissue (e.g. muscle, blood, central nervous system tissue, or renal tissue) from a human patient suspected of having a mitochondrial disease. The biological sample can also include cells from a human lymphoblast cell culture.

The biological sample can be a test sample obtained from a human patient suspected of having a mitochondrial disease. The method can further include subjecting a control sample to steps (a) through (d) wherein a control DHPLC elution profile is generated, and comparing the test sample DHPLC elution profile with the control DHPLC elution profile. Example of suitable control samples include: tissue from an individual not afflicted a mitochondrial disease; standard reference material SRM2392; cells from human lymphoblast cell culture line CHR; cells from human lymphoblast cell culture line 9947A; cells from human lymphoblast cell culture line K562. The DNA used in the preferred method can be total DNA from human lymphoblast cell culture line 9947A; total DNA from human lymphoblast cell culture line CHR; or a mixture of total DNA from human lymphoblast cell culture line 9947A and total DNA from human lymphoblast cell culture line CHR.

Also in the preferred method, the biological sample can be a test sample, and in step (d) a test sample DHPLC elution profile is generated. The method can further include subjecting a control sample to steps (a) through (d) wherein a control DHPLC elution profile is generated, and comparing the test sample DHPLC elution profile with the control DHPLC elution profile, wherein one or more differences (such as differences in peak number, or peak height) between the test sample DHPLC elution profile and the control DHPLC elution profile is indicative of the presence of at least one heteroduplex. For example, the test sample can be tissue from a human patient suspected of having a mitochondrial disease and the control sample can be tissue from an individual not afflicted with a mitochondrial disease.

In an embodiment of the method, the primer pairs are selected from a group consisting of forward primers and their respective reverse primers, wherein the forward primers consist of

SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35,

and wherein the reverse primers consist of

SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, and SEQ ID NO:36.

In another aspect, the invention provides a composition comprising the product of any one or more of step (a), step (b), and step (c) in claim 1. The biological sample can include cells from a lymphoblast cell culture line (e.g. CHR, 997A).

In yet another aspect, the invention concerns DHPLC profiles obtained during DHPLC analysis in step (d). The profiles can be obtained during analysis of lymphoblase tissue (e.g. CHR or 997A), or any other tissue.

In still another aspect, the invention provides kits for detecting mutations in the entire human mitochondrial genome. The kit can include: a) pre-selected pairs of primers for amplifying the entire genome by the polymerase chain reaction, wherein the pre-selected pairs of primers are selected such that amplicons obtained using the primers comprise overlapping segments of the entire mitochondrial genome, each of the primers in the kit in a separate container; and, b) one or more pre-selected restriction enzymes for cleaving amplification products obtained using the primers, wherein the enzymes are selected such that, for each of the primer pairs, the DNA products after the amplifying and the cleaving are between about 50 base pairs and about 700 base pairs in length, each of the restriction enzymes in the kit in a separate container. The DNA products after the amplifying and the cleaving are preferably between about 100 base pairs and about 600 base pairs in length.

In a preferred kit, the pairs of primers are selected from a group consisting of forward primers and their respective reverse primers as indicated hereinabove.

A kit of the invention can include restriction enzymes. Examples include AluI, DdeI, HaeIII, MboI, MspI, BfaI, NlaIII, HpaII, TaqI, HinfI, HphI, SfaNI, and DpnII. Preferably the restriction enzymes are selected from the group consisting of at least one of MboI, HaeIII, DdeI, MspI, and AluI. Preferably, the restriction enzymes each require about the same reaction temperature for optimal activity. A kit can also include a reverse phase column for separating double stranded DNA by denaturing high performance liquid chromatography. A kit can also include one or more of: a monolithic reverse phase column for separating double stranded DNA by denaturing high performance liquid chromatography; a disc or cartridge having hydrophobic separation surfaces for separating double stranded DNA; or a chromatography system for performing denatur;ing high performance liquid chromatography.

In other embodiments, a kit of the invention can include at least one DNA polymerase, such as a DNA polymerase having proofreading capability (a proofreading polymerase). Examples of suitable polymerase include one or more of Pho, Taq, and Pfu.

A kit can also include control DNA corresponding to the entire mitochondrial genome. Example of control DNA include DNA from cells of lymphoblast cell culture line CHR; DNA from cells of lymphoblast cell culture line 9947; DNA obtained from tissue of an individual who is not afflicted with mitochondrial disease; standard reference material SRM 2392; tissue of an individual (preferably maternally related to the test subject) who is not afflicted with mitochondrial disease.

A kit can include a control sample, such as cells from human lymphoblast cell culture line (e.g. CHR or 9947A).

In other embodiments, there is provide a kit for detecting mutations in the entire human mitochondrial genome. The kit can include: a set of pre-selected pairs of primers for amplifying the entire genome by the polymerase chain reaction, wherein the pre-selected pairs of primers are selected such that amplicons obtained using the primer pairs include overlapping segments of the entire mitochondrial genome, each of the primers in a separate container. The primer pairs are preferably selected such that the number of separate amplicons are in the range of about 15 to about 25. Each of the separate amplicons overlaps with its two neighboring amplicons, one at each end. The length of the overlap is preferably in the range of about 50 to about 1000 base pairs. The primers can be selected from a group consisting of forward primers and their respective reverse primers as indicated hereinabove, with each primer in a separate container. The kit can further include one or more pre-selected restriction enzymes.

In another aspect, the invention concerns a method for detecting the presence of one or more mutations in the entire human mitochondrial genome, wherein the genome is present in a test biological sample. The method preferably includes: (a) amplifying DNA from the test sample by polymerase chain reaction using a plurality of pre-selected primer pairs, wherein separate amplicons are generated for each primer pair, wherein the primer pairs are selected such that the amplicons comprise overlapping segments of the entire mitochondrial genome, wherein the amplicons comprise fragments that are greater than a size that is suitable for analysis by denaturing high performance liquid chromatography; (b) cleaving at least one of the amplicons using one or more pre-selected restriction enzymes, wherein the enzymes are selected such that for each of the separate amplicons the DNA products after the amplifying and the cleaving are within a size range that is suitable for analysis by denaturing high performance liquid chromatography; (c) denaturing and re-annealing the separate amplicons of step (b); (d) for each of the separate amplicons, analyzing the product of step (c) using denaturing high performance liquid chromatography, wherein the presence of the one or more mutations is confirmed if at least one heteroduplex is detected. The method preferably includes confirming that the cleaving is complete prior to step (d). The size range that is suitable for analysis by denaturing high performance liquid chromatography is between about 50 base pairs and about 1000 base pairs, and more preferably between about 100 base pairs and 600 base pairs. The amplicons in step (a) the DNA fragments that are greater than a size than is suitable for analysis by denaturing high performance liquid chromatography can include fragments that are at least about 1000 base pairs.

In another embodiment of the method of the invention concerns a method for detecting the presence of one or more mutations in the entire human mitochondrial genome, wherein the genome is present in a biological sample. The method includes: (a) amplifying DNA from the biological sample by polymerase chain reaction using a plurality of pre-selected primer pairs, wherein separate amplicons are generated from each primer pair, wherein the primer pairs are selected such that the amplicons comprise overlapping segments of the entire mitochondrial genome; (b) cleaving at least one of the separate amplicons using one or more pre-selected restriction enzymes, wherein the enzymes are selected such that, for each of the separate amplicons, the DNA products obtained after the amplifying and cleaving are between about 50 base pairs and about 700 base pairs in length; (c) for each of the separate amplicons, denaturing and re-annealing the separate amplicons of step (b); (d) for each of the separate amplicons, analyzing the product of step (c) using denaturing high performance liquid chromatography, wherein the presence of one or more mutations is confirmed if at least one heteroduplex is detected.

In yet a further aspect, the invention concerns a method for detecting the presence of one or more mutations in the entire human mitochondrial genome, wherein the genome is present in a biological sample. In one embodiment, the method comprises:(a) amplifying DNA from the biological sample by polymerase chain reaction using a plurality of pre-selected primer pairs, wherein separate amplicons are generated from each primer pair, wherein the primer pairs are selected such that the amplicons comprise overlapping segments of the entire mitochondrial genome; (b) for each of the separate amplicons, denaturing and re-annealing the separate amplicons of step (a); (c) cleaving at least one of the separate amplicons using pre-selected restriction enzymes, wherein the enzymes are selected such that, for each of the separate amplicons, the DNA products obtained after the amplifying and the cleaving are between about 100 base pairs and about 600 base pairs in length; and (d) for each of the separate amplicons, analyzing the product of step (c) using denaturing high performance liquid chromatography, wherein the presence of one or more mutations is confirmed if at least one heteroduplex is detected.

### BRIEF DESCRIPTION OF THE DRAWINGS

FIG. 1 shows a schematic representation of a hybridization to form homoduplex and heteroduplex DNA molecules and the DHPLC separation profile of the molecules.

FIG. 2 shows elution profiles obtained under non-denaturing elution conditions and illustrates restriction fragment length polymorphism in an amplified segment of mtDNA.

FIG. 3 illustrates detection of heteroduplex molecules in restriction digest fragments of an amplified first segment of mtDNA.

FIG. 4 illustrates detection of heteroduplex molecules in restriction digest fragments of an amplified second segment of mtDNA.

FIG. 5 illustrates detection of heteroduplex molecules in restriction digest fragments of an amplified third segment of mtDNA.

FIG. 6 illustrates detection of heteroduplex molecules in restriction digest fragments of an amplified fourth segment of mtDNA from two different cell lines and eluted at a first temperature.

FIG. 7 illustrates detection of heteroduplex molecules in the fragments as described in FIG. 6 and eluted at a second temperature.

FIG. 8 illustrates detection of heteroduplex molecules in the fragments as described in FIG. 6 and eluted at a third temperature.

FIG. 9 illustrates detection of heteroduplex molecules in the fragments as described in FIG. 6 and eluted at a fourth temperature.

FIG. 10 illustrates detection of heteroduplex molecules by DHPLC in restriction digest fragments and showing the effect of mixing in various proportions the restriction digest fragments obtained from different from different cell lines.

FIG. 11 shows an overlay of selected elution profiles from FIG. 10 and within a selected range of elution time.

### DETAILED DESCRIPTION OF THE INVENTION

In a general aspect, the invention concerns a method for detection of mutations in the entire human mitochondrial genome in a biological sample which contains mitochondria. The method generally entails:

(1) PCR amplification of the entire genome using pairs of PCR primers which are selected to generate separate amplification products (i.e. separate amplicons) that overlap and that span said entire genome, and wherein said separate amplicons include fragments that are greater than a size that is suitable (i.e. amenable) for analysis by DHPLC,

(2) cleaving at least some of the separate amplicons with at least one restriction enzyme such that for each separate amplicon, the DNA fragments produced are within a size range whereby the DNA fragments are suitable for analysis by DHPLC,

(3) verifying that said cleaving is complete,

(4) denaturing and re-annealing the product of step (3), and

(5) for each separate amplicon, analyzing the DNA products of step (4) by DHPLC in order to detect the presence of one or more heteroduplex molecule, wherein the presence of a heteroduplex molecule indicates the presence of a mutation. Preferred embodiments of the method are described in more detail hereinbelow.

Most of the mtDNA present in an individual is derived from the mtDNA contained within the ovum at the time of the individual's conception. Mutations in mtDNA sequence which affect all copies of mtDNA in an individual are known as homoplasmic. Mutations which affect only some copies of mtDNA are known as heteroplasmic and vary between different mitochondria in the same individual.

Each cell in an individual can contain hundreds of mitochondria and each mitochondria can contain multiple copies of the mitochondrial genome. Cells can harbor mixtures of mutant and normal mtDNA (heteroplasmy). During germ-line division (meiosis), mutant and normal mitochondria are randomly segregated into daughter cells. Random segregation of mitochondria during meiosis assures that the proportion of mutant to normal mitochondria within a daughter cell will vary. Because the severity of mitochondrial disease is a product of the nature of the mtDNA mutation, i.e., not all mutations will have a similar impact on function and the proportion of mutant mitochondria in a cell, random segregation of mtDNA causes mitochondrial diseases to appear sporadically in families with variable phenotypes. Offspring derived from a daughter cell acquiring a predominance of normal mitochondria will not express the disease whereas offspring derived from a daughter cell acquiring a predominance of mutant mitochondria will be severely affected. Gradations between these two extremes are also observed.

Mitochondria are unique cytoplasmic organelles distributed in all cells whose principal function is to generate energy-rich ATP molecules necessary for driving cellular biochemical processes. Mitochondria contain their own DNA that is separate and distinct from chromosomal DNA. Mitochondrial DNA (mtDNA) encodes exclusively for a number of critical protein subunits of the electron transport chain and the structural rRNAs and tRNAs necessary for the expression of these proteins. Unlike chromosomal DNA, each cell contains 1 to 10,000 copies of mtDNA. Cells can harbor mixtures of wild-type and mutant mtDNA (heteroplasmy). Mitochondrial genes are dynamic and the mtDNA genotype can drift towards increased mtDNA mutational burden in heteroplasmic cellular populations. The metabolic phenotype can deteriorate with time under these conditions, and can result in disease manifestation once the mutational burden exceeds a critical threshold in effected tissue, leading to bioenergetic failure and eventually cell death.

In the present invention, double stranded DNA is referred to as a duplex. When the base sequence of one strand is entirely complementary to base sequence of the other strand, the duplex is called a homoduplex. When a duplex contains at least one base pair which is not complementary, the duplex is called a heteroduplex.

Many different types of DNA mutations are known. Examples of DNA mutations include, but are not limited to, “point mutation” or “single base pair mutations” wherein an incorrect base pairing occurs. The most common point mutations comprise “transitions” wherein one purine or pyrimidine base is replaced for another and “transversions” wherein a purine is substituted for a pyrimidine (and visa versa). Such “insertions” or “deletions” are also known as “frameshift mutations”. Although they occur with less frequency than point mutations, larger mutations affecting multiple base pairs can also occur and may be important. A more detailed discussion of mutations can be found in U.S. Pat. No. 5,459,039 to Modrich (1995), and U.S. Pat. No. 5,698,400 to Cotton (1997).

Alterations in a DNA sequence which are benign or have no negative consequences are sometimes called “polymorphisms”. In the present invention, any alterations in the DNA sequence, whether they have negative consequences or not, are called “mutations”. It is to be understood that the method of this invention has the capability to detect mutations regardless of biological effect or lack thereof. For the sake of simplicity, the term “mutation” will be used throughout to mean an alteration in the base sequence of a DNA strand compared to a reference strand. It is to be understood that in the context of this invention, the term “mutation” includes the term “polymorphism” or any other similar or equivalent term of art.

“Reversed phase support” refers to a stationary support (including the base material and any chemically bonded phase) for use in liquid chromatography, particularly high performance liquid chromatography (HPLC), which is less polar (e.g., more hydrophobic) than the starting mobile phase.

“Ion-pair (IP) chromatography” refers to a chromatographic method for separating samples in which some or all of the sample components contain functional groups which are ionized or are ionizable. Ion-pair chromatography is typically carried out with a reversed phase column in the presence of an ion-pairing reagent.

“Ion-pairing reagent” is a reagent which interacts with ionized or ionizable groups in a sample to improve resolution in a chromatographic separation. An “ion-pairing agent” refers to both the reagent and aqueous solutions thereof An ion-pairing agent is typically added to the mobile phase in reversed phase liquid chromatography for optimal separation. The concentration and hydrophobicity of an ion-pairing agent of choice will depend upon the number and types (e.g., cationic or anionic) of charged sites in the sample to be separated.

“Primer” refers to an oligonuleotide, whether occurring naturally as in a purified restriction digest or produced synthetically, which is capable of acting as a point of initiation of synthesis when placed under conditions in which synthesis of a primer extension product that is complementary to a target nucleic acid strand is induced, i.e., in the presence of nucleotides and an agent for polymerization (such as a DNA polymerase) and at a suitable temperature and pH. The primer is preferably single stranded for maximum efficiency in amplification. Preferably, the primer is an oligodeoxyribonucleotide. The primer must be sufficiently long to prime the synthesis of extension products (referred to herein as “PCR products” and “PCR amplicons”) in the presence of the polymerization agent. Primers are preferably selected to be “substantially” complementary to a portion of the target nucleic acid sequence to be amplified. This typically means that the primer must be sufficiently complementary to hybridize with its respective portion of the target sequence. For example, a primer may include a non-complementary nucleotide portion at the 5′ end of the primer, with the remainder of the primer being complementary to a portion of the target sequence. Alternatively, non-complementary bases or longer sequences can be interspersed into the primer, provided that the primer sequence has sufficient complementarity with a portion of the target sequence to hybridize therewith, and thereby form a template for synthesis of the extension product.

A “homoduplex” is defined herein to include a double stranded DNA fragment wherein the bases in each strand are complementary relative to their counterpart bases in the other strand. “Homoduplex molecules” are typically composed of two complementary nucleic acid strands.

A “heteroduplex” is defined herein to include a double stranded DNA fragment wherein at least one base in each strand is not complementary to at least one counterpart base in the other strand. “Heteroduplex molecules” are typically composed of two complementary nucleic acid strands (e.g., DNA), where the strands have less than 100% sequence complementarity. Since at least one base pair in a heteroduplex is not complementary, it takes less energy to separate the bases at that site compared to its fully complementary base pair analog in a homoduplex. This results in the lower melting temperature at the site of a mismatched base of a heteroduplex compared to a homoduplex. A heteroduplex can be formed by annealing of two nearly complementary sequences. A heteroduplex molecule that is “partially denatured” under a given set of chromatographic conditions refers to a molecule in which several complementary base pairs of the duplex are not hydrogen-bond paired, such denaturing typically extending beyond the site of the base-pair mismatch contained in the heteroduplex, thereby enabling the heteroduplex to be distinguishable from a homoduplex molecule of essentially the same size. In accordance with the present invention, such denaturing conditions may be either chemically (e.g., resulting from pH conditions) or temperature-induced, or may be the result of both chemical and temperature factors.

“Mitochondrial disease” is defined to include a medical condition caused by abnormal mitochondria. Such diseases encompass a large assemblage of clinical problems, commonly involving tissues that have high energy requirements such as heart, muscle and the renal and endocrine systems. The genetic and molecular complexities of these diseases, which typically display an array of inheritance patterns, have been studied intensively over the past decade (Wallace, Science 283:1482-1488 (1999)). Examples of mitochondrial diseases include mitochondrial myopathy, limb-girdle-type myopathy, cardiomyopathy, Leigh syndrome, Leber's hereditary optic neuropathy, chronic progressive external ophthalmopelia, Alzheimer's disease, and Kearns-Sayre Syndrome.

The term “hybridization” refers to a process of heating and cooling a double stranded DNA (dsDNA) sample, e.g., heating to 95° C. followed by slow cooling. The heating process causes the DNA strands to denature. Upon cooling, the strands re-combine, or anneal, into duplexes.

“Heteroplasmic” is defined to include a mixture of wild-type and mutant mtDNA in the same tissue or cell.

“Homoplasmic” is defined to include the presence of a single type of mtDNA in a tissue or cell.

“Scanning” is defined herein to include the detection of any sequence-modifying mutation in a fragment without prior knowledge of its position or nature. Scanning techniques are frequently applied in the context of genetic variation discovery, as well as being a precursor to scoring, particularly when a very small number of a large pool of potential mutations is being subjected to detection. Scoring refers to the incontrovertible confirmation that a particular, predefined mutation or mutations are present within a sequence.

A “biological sample” is a sample of material derived from an organism.

As used herein “obtaining” a sample that includes, or that may include, an analyte polynucleotide can mean either obtaining from a biological subject such as a human, or obtaining from a reagent depository, such as a commercial vendor. When a sample is obtained from an animal or a human it will be understood that any number of appropriate means familiar to those having ordinary skill in the art can be employed. For example, if a blood sample is obtained, it can be obtained either by drawing blood through venepuncture, but also can be obtained as a forensic sample.

An “amplicon” is a polynucleotide product generated in an amplification reaction.

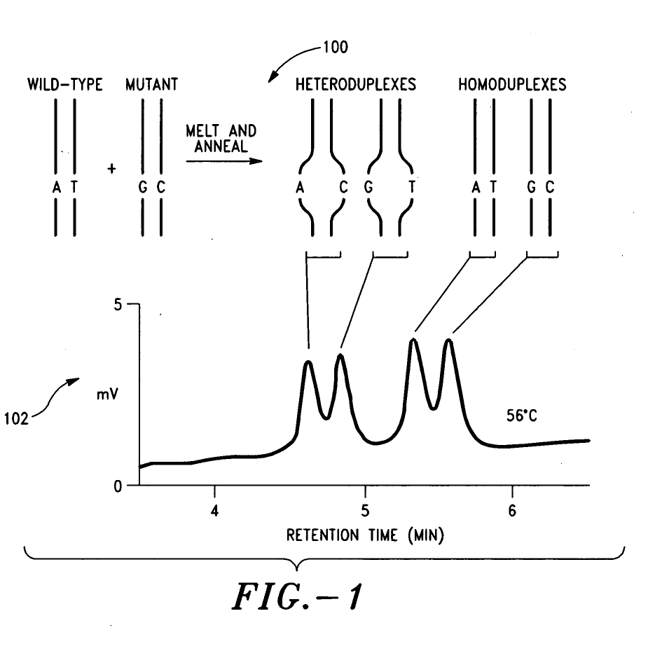

The term “DHPLC elution profile” is defined herein to include a separation chromatogram from a DHPLC analysis. If the injected sample contains heteroduplex and homoduplex molecules, the DHPLC elution profile shows the separation, or partial separation, of heteroduplexes from homoduplexes. Such separation profiles are characteristic of samples which contain mutations or polymorphisms and have been hybridized prior to being separated by DHPLC. The DHPLC separation chromatogram 102 shown in FIG. 1 exemplifies a mutation separation profile as defined herein.

The biological sample can be obtained from any tissue, and can include a test sample obtained from a living or deceased individual. Examples of such tissue include muscle, central nervous system tissue (e.g. brain), heart, endocrine system, kidney, liver, and blood. In practice, a test sample is obtained from a test subject, such as a patient who is suspected of having a mitochondrial disease. A control sample is processed in the same way as the test sample and can provide a basis for comparison. A control sample can be obtained from a person who is not afflicted with a mitochondrial disease, preferably a person having the same maternal lineage (e.g. a sibling). Alternatively, a control sample can be obtained from a non-affected tissue, such as blood, from the same patient who is suspected of having a mitochondrial disease. The control sample can also be obtained from a cell culture. Examples include cells from a human lymphoblast cell culture line, examples of which include CHR (product no. CCL-243; ATCC, Rockville, Md.), and 9947A (product no. DD1001; Promega, Madison Wis.). The mtDNA of these standard cells lines has been fully sequenced, and thus these cell lines can be used as positive control samples when analyzing test samples.

A DNA standard can provide a basis for comparison with DNA obtained from a test sample. A Standard Reference Material (SRM 2392) has been established to provide researchers with a well-characterized source of mitochondrial DNA for sequencing, forensic identifications, medical diagnostics, and mutation detection studies (Levin, B. C. et al. Genomics 55, 135-146 (1999)). The DNA extracted from cell lines, such CHR and 9947A, can also be used as a DNA standard. In some cases, a mixture of DNA extracted from such cell lines can be used as a positive control to assess the performance of the method, as described hereinbelow.

DHPLC elution profiles (i.e. chromatograms) obtained from the separation of double stranded DNA using DHPLC are highly reproducible (U.S. Pat. No. 6,287,822). Thus, the elution profiles obtained from the DHPLC analysis of standard cell lines can be used as standard DHPLC elution profiles (i.e. reference DHPLC elution profiles) which can be compared to DHPLC sample elution profiles obtained from test samples.

In a preferred embodiment of the method, total genomic DNA is extracted from a test sample from a patient suspected of having a mitochondrial disease.

Many techniques and methods have been described in the literature for the purpose of isolating nucleic acids from blood, cells or target tissue. Typically, methods used to obtain DNA utilize detergent action or mechanical treatment for disruption of cells, followed by enzymatic digestion of the protein contaminants with proteases such as Pronase and Proteinase K (see, e.g., Molecular Cloning: A Laboratory Manual, Sambrook, J. et al. Eds, Cold Spring Harbor Press (1989)). The nucleic acids are then purified by organic extraction with phenol/chloroform, followed by ethanol precipitation of the DNA from the aqueous phase. Other methods of DNA extraction from various tissue sources involve the use of chaotropic salts such as guanidinium isothiocyanate and guanidine hydrochloride.

Adaptations of the basic approaches outlined above are commonly used for DNA isolation from blood. In a simplification of these procedures, DNA can be obtained from small volumes of blood (about 5 μl) by boiling in water in the presence of chelating agents such as Chelex-100 (Bio-Rad Laboratories, Richmond, Calif.) and used in PCR reactions (see, e.g., Winberg, G., PCR Methods and Application, 1:72-74 (1991)). When obtaining DNA from blood, the extraction method can include a sedimentation procedure for separating erythrocytes from lymphocytes and platelets, and extraction of the DNA from the buffy coat fraction by boiling in water (U.S. Pat. No. 6,027,883).

The entire human mtDNA sequence has been determined (Anderson et al. Nature 290:457-465 (1981)). Functions and gene products have been assigned and a human mitochondrial genome database, MITOMAP, has been established (Nuc. Acids Res. 26:112-115 (1998); Wallace et al. Report of the committee on human mitochondrial DNA. In Cuticchia, ed., Human gene mapping 1995: a compendium. Johns Hopkins University Press, Baltimore, pp 910-954 (1995) (available on the World Wide Web at http://www.gen.emory.edu/mitomap.html)). MITOMAP provides a standardized system for numbering the base sequence of mtDNA. This sequence is used in the design of PCR primers and restriction enzymes, as described hereinbelow.

The present invention involves nucleic acid amplification procedures, such as PCR, which involve chain elongation by a DNA polymerase. There are a variety of different PCR techniques which utilize DNA polymerase enzymes, such as Taq polymerase. See PCR Protocols: A Guide to Methods and Applications. (Innis, M, Gelfand, D., Sninsky, J. and White, T., eds.), Academic Press, San Diego (1990) for detailed description of PCR methodology. PCR is also described in detail in U.S. Pat. No. 4,683,202 to Mullis (1987); Eckert et al., The Fidelity of DNA polymerases Used In The Polymerase Chain Reactions, McPherson, Quirke, and Taylor (eds.), “PCR: A Practical Approach”, IRL Press, Oxford, Vol. 1, pp. 225-244; Current Protocols in Molecular Biology, Ausubel et al. eds. John Wiley & Sons (1995), Chapter 15; and Andre, et. al., GENOME RESEARCH, Cold Spring Harbor Laboratory Press, pp. 843-852 (1977).

In a typical PCR protocol, a target nucleic acid, two oligonucleotide primers (one of which anneals to each strand), nucleotides, polymerase and appropriate salts are mixed and the temperature is cycled to allow the primers to anneal to the template, the DNA polymerase to elongate the primer, and the template strand to separate from the newly synthesized strand. Subsequent rounds of temperature cycling allow exponential amplification of the region between the primers.

There are a variety of different DNA polymerase enzymes that can be used in the invention, although proof-reading polymerases are preferred. DNA polymerases useful in the present invention may be any polymerase capable of replicating a DNA molecule. Preferred DNA polymerases are thermostable polymerases, which are especially useful in PCR. Thermostable polymerases are isolated from a wide variety of thermophilic bacteria, such as Thermus aquaticus (Taq), Thermus brockianus (Tbr), Thermus flavus (Tfl), Thermus ruber (Tru), Thermus thermophilus (Tth), Thermococcus litoralis (Tli) and other species of the Thermococcus genus, Thermoplasma acidophilum (Tac), Thermotoga neapolitana (Tne), Thermotoga maritima (Tma), and other species of the Thermotoga genus, Pyrococcus furiosus (Pfu), Pyrococcus horikoshii (Pho), Pyrococcus woesei (Pwo) and other species of the Pyrococcus genus, Bacillus sterothermophilus (Bst), Sulfolobus acidocaldarius (Sac) Sulfolobus solfataricus (Sso), Pyrodictium occultum (Poc), Pyrodictium abyssi (Pab), and Methanobacterium thermoautotrophicum (Mth), and mutants, variants or derivatives thereof.

Several DNA polymerases are known in the art and are commercially available (e.g., from Boehringer Mannheim Corp., Indianapolis, Ind.; Life Technologies, Inc., Rockville, Md; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif.; Transgenomic, Omaha, Nebr.). Preferably the thermostable DNA polymerase is selected from the group of Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT™, DEEPVENT™, PFUTurbo™, AmpliTaq®, and active mutants, variants and derivatives thereof. It is to be understood that a variety of DNA polymerases may be used in the present invention, including DNA polymerases not specifically disclosed above, without departing from the scope or preferred embodiments thereof.

The PCR preferably utilizes buffers and other solutions that are compatible with DHPLC analysis as described in U.S. patent application Ser. No. 10/126,848, filed Apr. 19, 2002. The PCR buffers, enzymes preparations, and other solutions minimize, or preferably exclude, BSA, mineral oil, formamide, polyethylene glycol, detergents such as Triton X-100, NP40, Tween 20, sodium dodecyl sulfate and sodium lauryl sulfate. Other reagents, such as those commonly used in the purification of DNA, such as proteases, solvents, nucleases, phenol, guanidinium, etc., are preferably removed in a final ethanol precipitation and wash step prior to PCR. Excess EDTA, isopropanol, or iso-amyl alcohol are also preferably removed. Examples of suitable proof reading enzyme preparations includes Pho polymerase (available as Optimase™ polymerase (Transgenomic) and AccuType™ DNA polymerase (Stratagene).

In a typical PCR protocol, a target nucleic acid, two oligonucleotide primers (one of which anneals to each strand), nucleotides, polymerase and appropriate salts are mixed and the temperature is cycled to allow the primers to anneal to the template, the DNA polymerase to elongate the primer, and the template strand to separate from the newly synthesized strand. Subsequent rounds of temperature cycling allow exponential amplification of the region between the primers.

In another aspect, the invention concerns the design of PCR primers to be used in analyzing the entire mitochondrial genome. Oligonucleotide primers useful in the present invention may be any oligonucleotide of two or more nucleotides in length. Preferably, PCR primers are about 15 to about 30 bases in length, and are not palindromic (self-complementary) or complementary to other primers that may be used in the reaction mixture. Oligonucleotide primers are oligonucleotides used to hybridize to a region of a target nucleic acid to facilitate the polymerization of a complementary nucleic acid. Any primer may be synthesized by a practitioner of ordinary skill in the art or may be purchased from any of a number of commercial venders (e.g., from Boehringer Mannheim Corp., Indianapolis, Ind.; New England Biolabs, Inc., Beverley, Mass.; Pharmacia LKB Biotechnology, Inc., Piscataway, N.J.). It will be recognized that the PCR primers can include covalently attached groups, such as fluorescent tags. U.S. Pat. No. 6,210,885 describes the use of such tags in mutation detection by DHPLC. It is to be understood that a vast array of primers may be useful in the present invention, including those not specifically disclosed herein, without departing from the scope or preferred embodiments thereof.

Buffering agents and salts are used in the PCR buffers and storage solutions of the present invention to provide appropriate stable pH and ionic conditions for nucleic acid synthesis, e.g., for DNA polymerase activity, and for the hybridization process. A wide variety of buffers and salt solutions and modified buffers are known in the art that may be useful in the present invention, including agents not specifically disclosed herein. Preferred buffering agents include, but are not limited to, TRIS, TRICINE, BIS-TRICINE, HEPES, MOPS, TES, TAPS, PIPES, CAPS. Preferred salt solutions include, but are not limited to solutions of; potassium acetate, potassium sulfate, ammonium sulfate, ammonium chloride, ammonium acetate, magnesium chloride, magnesium acetate, magnesium sulfate, manganese chloride, manganese acetate, manganese sulfate, sodium chloride, sodium acetate, lithium chloride, and lithium acetate.

An important aspect of the invention includes selecting a plurality of PCR primer pairs (i.e. sets of forward and reverse primers) that span the entire mitochondrial genome. The primer pairs are selected so that there is overlap at the ends of adjacent amplicons. In the Examples hereinbelow, each primer pair is identified by an individual mitochondrial (MT) set number.

In a preferred embodiment of the invention, a plurality of primer pairs are designed in order to separately amplify adjacent regions of the entire mitochondrial genome. The number of amplicons can be in the range of 1 to 70, and is preferably in the range of 10 to 50, and most preferably in the range of 15 to 25. The primers are also designed so that there is an overlap between adjacent regions being amplified. Thus, each separate amplicon has sequences at its ends which overlap (i.e. are identical with) the end sequences of its two neighboring amplicons. The resulting separate amplicons constitute overlapping segments of the entire mitochondrial genome.

The ends of the amplicons are preferably generated using synthetic DNA primers approximately 20 to 24 base pairs in length. However, any mutations (base changes) that occur “under” the primer (i.e. the complementary sequence where the primer anneals) will be missed by DHPLC analysis because the sequence of the primer itself (which primer is present in excess during PCR), and not the mutated sequence, will be amplified during the PCR.

In order to maximize the ability to detect mutations using the inventive method, Applicants have observed that the primer pairs can be selected so that the overlap at the ends of each amplicon is at least about 50 base pairs, preferably in the range of about 50 base pairs to about 1000 base pairs, and optimally in the range of about 60 to about 500 base pairs.

It is advantageous to minimize the number of primer pairs that are to be used, since this can lower the cost involved in obtaining synthetic oligo primers. In general, however, the lower the number of primer pairs, the longer will be the average length of the DNA amplicons.

Applicants observed that double stranded DNA fragments that exceed about 1000 base pairs gave poor resolution when analyzed using DHPLC and thus were not suitable for analysis via DHPLC. In DHPLC, the DNA fragment size range is operationally between about 50 and about 1000 base pairs, preferably between about 70 and about 1000 base pairs, and more preferably between about 70 and about 700 base pairs, and most preferably between about 150 and about 600 base pairs.

Another aspect of the method of the invention involves the use of restriction enzymes to cleave each separate amplicon into smaller fragments that are suitable for analysis by DHPLC. Multiple fragments could interfere with the ability to detect heteroduplex molecules during analysis by DHPLC. DHPLC produces chromatograms that typically show multiple adjacent peaks (up to four peaks in some cases, see FIG. 1), or shoulders on peaks, due to the formation of heteroduplex and homoduplex molecules. In a preferred method of the invention, after PCR amplification and restriction enzyme cleavage, DNA the fragments are subjected to hybridization in order to induce heteroduplex formation.

In the present invention, the primer pairs and restriction enzymes are preferably selected such that it is still possible to detect the presence of heteroduplex molecules even in the presence of multiple restriction fragments. In the design of primer pairs for mtDNA analysis, some of the amplicons can be designed to be in the size range of about 100 to about 600 base pairs, and therefore do not require digestion by restriction enzymes. For example, in analyzing mtDNA of standard cell lines (as described in the Examples hereinbelow), Applicants observed that that it was preferable to amplify the hypervariable regions (HV1, HV2) of mtDNA as fragments of about 500 base pairs that did not require cleavage by a restriction enzyme. These regions are known to be highly variable in sequence, and therefore would likely give restriction fragment polymorphism that would complicate the analysis. These regions also include many polymorphic base changes and therefore will test positive for the presence of a mutation in the method of the invention.

An important aspect of the instant invention concerns the effect of PCR amplification on heteroplasmic mtDNA. If the test sample is homoplasmic, then only homoduplexes are generated during PCR. However, if the test sample is heteroplasmic, then heteroduplexes are generated during the melting and annealing steps in PCR because the sample contains both normal and mutant double stranded DNA.

In DHPLC as routinely practiced, a corresponding wild type DNA fragment is typically added to the DNA sample fragment suspected of having a mutation prior to hybridization. However, in preparing mtDNA from a sample for determination of whether or not the sample is heteroplasmic, no corresponding wild type DNA fragment need be added. The DHPLC analysis of the mtDNA tests directly for the presence of heteroduplx molecules in the test sample, and thus provides an indication of whether or not the sample is heteroplasmic.

In an example of a hybridization procedure, analysis of hypothetical DNA fragments derived from heteroplasmic mtDNA is schematically illustrated in FIG. 1. Prior to injection of the mixture onto the separation column, the mixture is hybridized as shown in the scheme 100. The hybridization process creates two homoduplexes and two heteroduplexes. As shown in the mutation separation profile 102, the hybridization product was separated using DHPLC. The two lower retention time peaks represent the two heteroduplexes and the two higher retention time peaks represent the two homoduplexes. The two homoduplexes separate because the A-T base pair denatures at a lower temperature than the C-G base pair. Without wishing to be bound by theory, the results are consistent with a greater degree of denaturation in one duplex and/or a difference in the polarity of one partially denatured heteroduplex compared to the other, resulting in a difference in retention time on the reverse-phase separation column.

In the instant invention, it will be understood that a test sample from a heteroplasmic tissue will produce heteroduplexes without the addition of wild type DNA. If the test sample is homoplasmic, then no heteroduplexes will be observed.

In one embodiment of the instant invention, DNA obtained from a test sample is mixed with DNA from a control (e g. DNA obtained from a non-afflicted individual having the same maternal lineage, or DNA from a non-affected tissue). This method can be used, for example, in order to detect whether the test sample is homoplasmic for a mutation. In this embodiment, the DNA is PCR amplified, subjected to restriction enzyme cleavage, mixed, hybridized, and analyzed by DHPLC. If the test sample contains a mutation, then the hybridization product ideally includes both homoduplex and heteroduplex molecules. If no mutation is present, then the hybridization only produces homoduplex molecules.

In the selection of primer pairs in the instant invention, it is preferred to minimize the number of primer pairs that are to be used, since this can lower the cost involved in obtaining synthetic oligo primers. In general, the lower the number of primer pairs, the longer will be the length of the amplicons. Decreasing the number of amplicons increases average length of the amplicons.

However, Applicants observed that double stranded DNA fragments that exceed about 600-700 base pairs gave poor resolution when analyzed using DHPLC and thus were not suitable for analysis via DHPLC.

Another aspect of the method of the invention involves the use of restriction enzymes to cleave at least some of the separate amplicons from mtDNA amplification into smaller fragments that are suitable for analysis by DHPLC. In DHPLC, the DNA fragment size range is operationally between about 50 to about 1000 base pairs, preferably between about 70 to about 1000 base pairs, more preferably between about 50 to about 700 base pairs, and optimally between about 100 to about 600 base pairs.

However, the cleavage by restriction enzymes typically generates multiple fragments for each amplification. It was still possible to detect the presence of heteroduplex molecules even in the presence of multiple restriction fragments. Applicants determined that in order to detect mutations, the difference in length between restriction fragments could be at least 20 base pairs, preferably at least about 40 base pairs, more preferably at least about 100 base pairs, and optimally at least 300 base pairs. Maximizing the difference in size of the restriction fragments decreases the potential for peak overlap in the DHPLC elution profile.

Conventional software can be used to map restriction sites and fragments. An example of such a program is Gene Runner version 3.05 (Hastings Software, Inc., Hastings, N.Y.).

It is preferable to minimize the number of restriction enzymes required to digest the amplicons in order to lower the cost of the procedure. In addition, the use of restriction enzymes that all require the same digestion temperature (e.g. 37° C.) can simplify the restriction digest procedure. The position of restriction sites, and selection of restriction enzymes, were both considered in the design of the primer pairs as described herein. Primer pairs and restriction enzymes can be chosen to pre-select the fragment size, and therefore the spacing between peaks in the elution profile. The retention times of all double stranded DNA fragments can be predicted using software such as Wavemaker™ software (Transgenomic) or Star workstation software (Varian). These programs allow prediction of the retention time based on the length of a DNA fragment for a given set of elution conditions (U.S. Pat. Nos. 6,287,822 and 6,197,516; and in U.S. patent application Ser. No. 09/469,551 filed Dec. 22, 1999; and PCT publications WO99/07899 and WO 01/46687).

Evaluation of the restriction enzyme cleavage products is an important step in the inventive method for two reasons. Firstly, the highly polymorphic nature of the mtDNA sequence can result in base changes that cause the gain or loss of an enzyme recognition site. FIG. 2 illustrates an example of the gain of an AluI site in MT set # 18 in the K562 cell line (Example 4). It would be virtually impossible to correctly interpret the elution profile of this sample at elevated column temperatures. Secondly, this step is important to determine that no partial restriction enzyme products are present that might also lead to misinterpretation of the results.

This evaluation of the restriction products can be effected using a variety of separation methods, such as gel electrophoresis or capillary electrophoresis, and denaturing anion exchange chromatography. However a preferred method is separation at a non-denaturing column temperature (e.g. 50° C.) by IP-RP-HPLC as described herein.

The instant invention concerns chromatographic separation of DNA fragments for analysis of mutations in mtDNA. Recently, a chromatographic method called ion-pair reverse-phase high performance liquid chromatography (IP-RP-HPLC), also referred to as Matched Ion Polynucleotide Chromatography (MIPC), was introduced to effectively separate mixtures of double stranded polynucleotides, in general and DNA, in particular, wherein the separations are based on base pair length (Huber, et al., Chromatographia 37:653 (1993); Huber, et al., Anal. Biochem. 212:351 (1993); U.S. Pat. Nos. 5,585,236; 5,772,889; 5,972,222; 5,986,085; 5,997,742; 6,017,457; 6,030,527; 6,056,877; 6,066,258; 6,210,885; and U.S. patent application Ser. No. 09/129,105 filed Aug. 4, 1998.

As the use and understanding of IP-RP-HPLC developed it became apparent that when IP-RP-HPLC analyses were carried out at a partially denaturing temperature, i.e., a temperature sufficient to denature a heteroduplex at the site of base pair mismatch, homoduplexes could be separated from heteroduplexes having the same base pair length (Hayward-Lester, et al., Genome Research 5:494 (1995); Underhill, et al., Proc. Natl. Acad. Sci. U.S.A 93:193 (1996); Doris, et al., DHPLC Workshop, Stanford University, (1997)). These references and the references contained therein are incorporated herein in their entireties. Thus, the use of denaturing high performance liquid chromatography (DHPLC) was applied to mutation detection (Underhill, et al., Genome Research 7:996 (1997); Liu, et al., Nucleic Acid Res., 26;1396 (1998)).

These chromatographic methods are generally used to detect whether or not a mutation exists in a test DNA fragment. Depending on the conditions, ion-pair reverse-phase high performance liquid chromatography (IP-RP-HPLC) separates double stranded polynucleotides by size or by base pair sequence and is therefore a preferred separation technology for detecting the presence of particular fragments of DNA of interest. The chromatographic profile can be in the form of a visual display, a printed representation of the data or the original data stream.

When mixtures of DNA fragments are mixed with an ion pairing agent and applied to a reverse phase separation column, they are separated by size, the smaller fragments eluting from the column first. IP-RP-HPLC, when performed at a temperature which is sufficient to partially denature a heteroduplex, is referred to as DHPLC. DHPLC is also referred to in the art as “Denaturing Matched Ion Polynucleotide Chromatography” (DMIPC).

Examples of suitable separation media are described in the following U.S. patents and patent applications: U.S. Pat. Nos. 6,379,889; 6,056,877; 6,066,258; 5,453,185; 5,334,310; U.S. patent application Ser. No. 09/493,734 filed Jan. 28, 2000; U.S. patent application Ser. No. 09/562,069 filed May 1, 2000; and in the following PCT applications: WO98/48914; WO98/48913; PCT/US98/08388; PCT/US00/11795.

An example of a suitable column based on a polymeric stationary support is the DNASep® column (Transgenomic). Examples of a suitable column based on a silica stationary support include the Microsorb Analytical column (Varian and Rainin) and “ECLIPSE dsDNA” (Hewlett Packard, Newport, Del.).

The length and diameter of the separation column, as well as the system mobile phase pressure and temperature, and other parameters, can be varied as is known in the art. An increase in the column diameter was found to increase resolution of fragments in IP-RP-HPLC and DHPLC (U.S. Pat. No. 6,372,142; WO 01/19485). Size-based separation of DNA fragments can also be performed using batch methods and devices as disclosed in U.S. Pat. Nos. 6,265,168; 5,972,222; and 5,986,085.

In DHPLC, the mobile phase contains an ion-pairing agent (i.e. a counter ion agent) and an organic solvent. Ion-pairing agents for use in the method include lower primary, secondary and tertiary amines, lower trialkylammonium salts such as triethylammonium acetate and lower quaternary ammonium salts. Typically, the ion-pairing reagent is present at a concentration between about 0.05 and 1.0 molar. Organic solvents for use in the method include solvents such as methanol, ethanol, 2-propanol, acetonitrile, and ethyl acetate.

In one embodiment, the mobile phase for carrying out the separation of the present invention contains less than about 40% by volume of an organic solvent and greater than about 60% by volume of an aqueous solution of the ion-pairing agent. In a preferred embodiment, elution is carried out using a binary gradient system.

Partial denaturation of heteroduplex molecules can be carried out in a variety of ways such as alteration of pH or salt concentration, use of denaturing agents, or elevation in temperature. Temperatures for carrying out the separation method of the invention are typically between about 40° and 70° C., preferably between about 55°-65° C. The preferred temperature is sequence dependent. In carrying out a separation of GC-rich heteroduplex and homoduplex molecules, a higher temperature is preferred.

A variety of liquid chromatography systems are available that can be used for conducting DHPLC. These systems typically include software for operating the chromatography components, such as pumps, heaters, mixers, fraction collection devices, injector. Examples of software for operating a chromatography apparatus include HSM Control System (Hitachi), ChemStation (Agilent), VP data system (Shimadzu), Millennium32 Software (Waters), Duo-Flow software (Bio-Rad), and Star workstation (Varian). Examples of preferred liquid chromatography systems for carrying out DHPLC include the WAVE® DNA Fragment Analysis System (Transgenomic) and the Varian ProStar Helix™ System (Varian).

In carrying out DHPLC analysis, the operating temperature and the mobile phase composition can be determined by trial and error. However, these parameters are preferably obtained by using software. Computer software that can be used in carrying out DHPLC is disclosed in the following patents and patent applications: U.S. Pat. Nos. 6,287,822; 6,197,516; U.S. patent application Ser. No. 09/469,551 filed Dec. 22, 1999; and in WO0146687 and WO0015778. Examples of software for predicting the optimal temperature for DHPLC analysis are disclosed by Jones et al. in Clinical Chem. 45:113-1140 (1999) and in the website having the address of http://insertion.stanford.edu/melt.html. And example of a commercially available software includes WAVEMaker™ software and Navigator™ software (Transgenomic).

Ion-Pairing Reversed-Phase Chromatography (IP-RPC) is a powerful form of chromatography used in the separation and analysis of polynucleotides, including DNA (both single and double stranded) and RNA (Eriksson et al., (1986) J. Chromatography 359:265-74). Most reported applications of IP-RPC have been in the context of high performance liquid chromatography (IP-RP-HPLC), but the technology can be accomplished using non-HPLC chromatography systems (U.S. patent application Ser. Nos. 09/318,407 and 09/391,963. Nevertheless, for the sake of simplicity much of the following description will focus on the use of IP-RP-HPLC, a particularly powerful and convenient form of IP-RPC. It is to be understood that this is not intended to limit the scope of the invention, and that generally the methods described can be performed without the use of HPLC, although this will in some cases lead to less than optimal results. IP-RPC is a form of chromatography characterized by the use of a reversed phase (i.e., hydrophobic) stationary phase and a mobile phase that includes an alkylated cation (e.g., triethylammonium) that is believed to form a bridging interaction between the negatively charged polynucleotide and non-polar stationary phase. The alkylated cation-mediated interaction of polynucleotide and stationary phase can be modulated by the polarity of the mobile phase, conveniently adjusted by means of a solvent that is less polar than water, e.g., acetonitrile. In general, a polynucleotide such as RNA is retained by the separation medium in the presence of counterion agent, and can be eluted by increasing the concentration of a non-polar solvent, Elution can be accomplished in the presence or absence of counterion agent. Performance is enhanced by the use of a non-porous separation medium, as described in U.S. patent application Ser. No. 5,585,236. MIPC, is described in U.S. Pat. Nos. 5,585,236, 6,066,258 and 6,056,877 and PCT Publication Nos. WO98/48913, WO98/48914, WO/9856797, WO98/56798, incorporated herein by reference in their entirety. MIPC is characterized by the preferred use of solvents and chromatographic surfaces that are substantially free of multivalent cation contamination that can interfere with polynucleotide separation. In the practice of the instant invention, a preferred system for performing MIPC separations is that provided by Transgenomic, Inc. under the trademark WAVE™.

Separation by IP-RP-HPLC, including MIPC, occurs at the non-polar surface of a separation medium. In one embodiment, the non-polar surfaces comprise the surfaces of polymeric beads. In an alternative embodiment, the surfaces comprise the surfaces of interstitial spaces in a molded polymeric monolith, described in more detail infra. For purposes of simplifying the description of the invention and not by way of limitation, the separation of polynucleotides using nonporous beads, and the preparation of such beads, will be primarily described herein, it being understood that other separation surfaces, such as the interstitial surfaces of polymeric monoliths, are intended to be included within the scope of this invention.

In general, in order to be suitable for use in IP-RP-HPLC a separation medium should have a surface that is either intrinsically non-polar or bonded with a material that forms a surface having sufficient non-polarity to interact with a counterion agent.

In one aspect of the invention, IP-RP-HPLC detection is accomplished using a column filled with nonporous polymeric beads having an average diameter of about 0.5-100 microns; preferably, 1-10 microns; more preferably, 1-5 microns. Beads having an average diameter of 1.0-3.0 microns are most preferred.

In a preferred embodiment of the invention, the chromatographic separation medium comprises nonporous beads, i.e., beads having a pore size that essentially excludes the polynucleotides being separated from entering the bead, although porous beads can also be used. As used herein, the term “nonporous” is defined to denote a bead that has surface pores having a diameter that is sufficiently small so as to effectively exclude the smallest DNA fragment in the separation in the solvent medium used therein. Included in this definition are polymer beads having these specified maximum size restrictions in their natural state or which have been treated to reduce their pore size to meet the maximum effective pore size required.

The surface conformations of nonporous beads of the present invention can include depressions and shallow pit-like structures that do not interfere with the separation process. A pretreatment of a porous bead to render it nonporous can be effected with any material which will fill the pores in the bead structure and which does not significantly interfere with the IP-RP-HPLC process.

Pores are open structures through which mobile phase and other materials can enter the bead structure. Pores are often interconnected so that fluid entering one pore can exit from another pore. Without intending to be bound by any particular theory, it is believed that pores having dimensions that allow movement of the polynucleotide into the interconnected pore structure and into the bead impair the resolution of separations or result in separations that have very long retention times.

Non-porous polymeric beads useful in the practice of the present invention can be prepared by a two-step process in which small seed beads are initially produced by emulsion polymerization of suitable polymerizable monomers. The emulsion polymerization procedure is a modification of the procedure of Goodwin, et al. (Colloid & Polymer Sci., 252:464-471 (1974)). Monomers which can be used in the emulsion polymerization process to produce the seed beads include styrene, alkyl substituted styrenes, alpha-methyl styrene, and alkyl substituted alpha-methyl styrene. The seed beads are then enlarged and, optionally, modified by substitution with various groups to produce the nonporous polymeric beads of the present invention.

The seed beads produced by emulsion polymerization can be enlarged by any known process for increasing the size of the polymer beads. For example, polymer beads can be enlarged by the activated swelling process disclosed in U.S. Pat. No. 4,563,510. The enlarged or swollen polymer beads are further swollen with a crosslinking polymerizable monomer and a polymerization initiator. Polymerization increases the crosslinking density of the enlarged polymeric bead and reduces the surface porosity of the bead. Suitable crosslinking monomers contain at least two carbon-carbon double bonds capable of polymerization in the presence of an initiator. Preferred crosslinking monomers are divinyl monomers, preferably alkyl and aryl (phenyl, naphthyl, etc.) divinyl monomers and include divinyl benzene, butadiene, etc. Activated swelling of the polymeric seed beads is useful to produce polymer beads having an average diameter ranging from 1 up to about 100 microns.

Alternatively, the polymer seed beads can be enlarged simply by heating the seed latex resulting from emulsion polymerization. This alternative eliminates the need for activated swelling of the seed beads with an activating solvent. Instead, the seed latex is mixed with the crosslinking monomer and polymerization initiator described above, together with or without a water-miscible solvent for the crosslinking monomer. Suitable solvents include acetone, tetrahydrofuran (THF), methanol, and dioxane. The resulting mixture is heated for about 1-12 hours, preferably about 4-8 hours, at a temperature below the initiation temperature of the polymerization initiator, generally, about 10° C.-80° C., preferably 30° C.-60° C. Optionally, the temperature of the mixture can be increased by 10-20% and the mixture heated for an additional 1 to 4 hours. The ratio of monomer to polymerization initiator is at least 100:1, preferably in the range of about 100:1 to about 500:1, more preferably about 200:1 in order to ensure a degree of polymerization of at least 200. Beads having this degree of polymerization are sufficiently pressure-stable to be used in HPLC applications. This thermal swelling process allows one to increase the size of the bead by about 110-160% to obtain polymer beads having an average diameter up to about 5 microns, preferably about 2-3 microns. The thermal swelling procedure can, therefore, be used to produce smaller particle sizes previously accessible only by the activated swelling procedure.

Following thermal enlargement, excess crosslinking monomer is removed and the particles are polymerized by exposure to ultraviolet light or heat. Polymerization can be conducted, for example, by heating of the enlarged particles to the activation temperature of the polymerization initiator and continuing polymerization until the desired degree of polymerization has been achieved. Continued heating and polymerization allows one to obtain beads having a degree of polymerization greater than 500.

For use in the present invention, packing material disclosed by U.S. Pat. No. 4,563,510 can be modified through substitution of the polymeric beads with alkyl groups or can be used in its unmodified state. For example, the polymer beads can be alkylated with 1 or 2 carbon atoms by contacting the beads with an alkylating agent, such as methyl iodide or ethyl iodide. Alkylation can be achieved by mixing the polymer beads with the alkyl halide in the presence of a Friedel-Crafts catalyst to effect electrophilic aromatic substitution on the aromatic rings at the surface of the polymer blend. Suitable Friedel-Crafts catalysts are well-known in the art and include Lewis acids such as aluminum chloride, boron trifluoride, tin tetrachloride, etc. The beads can be hydrocarbon substituted by substituting the corresponding hydrocarbon halide for methyl iodide in the above procedure, for example.

The term alkyl as used herein in reference to the beads useful in the practice of the present invention is defined to include alkyl and alkyl substituted aryl groups, having from 1 to 1,000,000 carbons, the alkyl groups including straight chained, branch chained, cyclic, saturated, unsaturated nonionic functional groups of various types including aldehyde, ketone, ester, ether, alkyl groups, and the like, and the aryl groups including as monocyclic, bicyclic, and tricyclic aromatic hydrocarbon groups including phenyl, naphthyl, and the like. Methods for alkyl substitution are conventional and well-known in the art and are not an aspect of this invention. The substitution can also contain hydroxy, cyano, nitro groups, or the like which are considered to be non-polar, reverse phase functional groups.

Non-limiting examples of base polymers suitable for use in producing such polymer beads include mono- and di-vinyl substituted aromatics such as styrene, substituted styrenes, alpha-substituted styrenes and divinylbenzene; acrylates and methacrylates; polyolefins such as polypropylene and polyethylene; polyesters; polyurethanes; polyamides; polycarbonates; and substituted polymers including fluorosubstituted ethylenes commonly known under the trademark TEFLON. The base polymer can also be mixtures of polymers, non-limiting examples of which include poly(styrene-divinylbenzene) and poly(ethylvinylbenzene-divinylbenzene). Methods for making beads from these polymers are conventional and well known in the art (for example, see U.S. Pat. No. 4,906,378). The physical properties of the surface and near-surface areas of the beads are the primary determinant of chromatographic efficiency. The polymer, whether derivatized or not, should provide a nonporous, non-reactive, and non-polar surface for the MIPC separation. In a particularly preferred embodiment of the invention, the separation medium consists of octadecyl modified, nonporous alkylated poly(styrene-divinylbenzene) beads. Separation columns employing these particularly preferred beads, referred to as DNASep® columns, are commercially available from Transgenomic, Inc.

A separation bead used in the invention can comprise a nonporous particle which has non-polar molecules or a non-polar polymer attached to or coated on its surface. In general, such beads comprise nonporous particles which have been coated with a polymer or which have substantially all surface substrate groups reacted with a non-polar hydrocarbon or substituted hydrocarbon group, and any remaining surface substrate groups endcapped with a tri(lower alkyl)chlorosilane or tetra(lower alkyl)dichlorodisilazane as described in U.S. Pat. No. 6,056,877.

The nonporous particle is preferably an inorganic particle, but can be a nonporous organic particle. The nonporous particle can be, for example, silica, silica carbide, silica nitrite, titanium oxide, aluminum oxide, zirconium oxide, carbon, insoluble polysaccharides such as cellulose, or diatomaceous earth, or any of these materials which have been modified to be nonporous. Examples of carbon particles include diamond and graphite which have been treated to remove any interfering contaminants. The preferred particles are essentially non-deformable and can withstand high pressures. The nonporous particle is prepared by known procedures. The preferred particle size is about 0.5-100 microns; preferably, 1-10 microns; more preferably, 1-5 microns. Beads having an average diameter of 1.0-3.0 microns are most preferred.

Because the chemistry of preparing conventional silica-based reverse phase HPLC materials is well-known, most of the description of non-porous beads suitable for use in the instant invention is presented in reference to silica. It is to be understood, however, that other nonporous particles, such as those listed above, can be modified in the same manner and substituted for silica. For a description of the general chemistry of silica, see Poole, Colin F. and Salwa K. Poole, Chromatography Today, Elsevier:New York (1991), pp. 313-342 and Snyder, R. L. and J. J. Kirkland, Introduction to Modem Liquid Chromatography, 2^{nd} ed., John Wiley & Sons, Inc.: New York (1979), pp. 272-278, the disclosures of which are hereby incorporated herein by reference in their entireties.

The nonporous beads of the invention are characterized by having minimum exposed silanol groups after reaction with the coating or silating reagents. Minimum silanol groups are needed to reduce the interaction of the DNA with the substrate and also to improve the stability of the material in a high pH and aqueous environment. Silanol groups can be harmful because they can repel the negative charge of the DNA molecule, preventing or limiting the interaction of the DNA with the stationary phase of the column. Another possible mechanism of interaction is that the silanol can act as ion exchange sites, taking up metals such as iron (III) or chromium (III). Iron (III) or other metals which are trapped on the column can distort the DNA peaks or even prevent DNA from being eluted from the column.

Silanol groups can be hydrolyzed by the aqueous-based mobile phase. Hydrolysis will increase the polarity and reactivity of the stationary phase by exposing more silanol sites, or by exposing metals that can be present in the silica core. Hydrolysis will be more prevalent with increased underivatized silanol groups. The effect of silanol groups on the DNA separation depends on which mechanism of interference is most prevalent. For example, iron (III) can become attached to the exposed silanol sites, depending on whether the iron (III) is present in the eluent, instrument or sample.

The effect of metals can only occur if metals are already present within the system or reagents. Metals present within the system or reagents can get trapped by ion exchange sites on the silica. However, if no metals are present within the system or reagents, then the silanol groups themselves can cause interference with DNA separations. Hydrolysis of the exposed silanol sites by the aqueous environment can expose metals that might be present in the silica core.

Fully hydrolyzed silica contains a concentration of about 8 μmoles of silanol groups per square meter of surface. At best, because of steric considerations, a maximum of about 4.5 μmoles of silanol groups per square meter can be reacted, the remainder of the silanol being sterically shielded by the reacted groups. Minimum silanol groups is defined as reaching the theoretical limit of or having sufficient shield to prevent silanol groups from interfering with the separation.

Numerous methods exist for forming nonporous silica core particles. For example, sodium silicate solution poured into methanol will produce a suspension of finely divided spherical particles of sodium silicate. These particles are neutralized by reaction with acid. In this way, globular particles of silica gel are obtained having a diameter of about 1-2 microns. Silica can be precipitated from organic liquids or from a vapor. At high temperature (about 2000° C.), silica is vaporized, and the vapors can be condensed to form finely divided silica either by a reduction in temperature or by using an oxidizing gas. The synthesis and properties of silica are described by R. K. Iler in The Chemistry of Silica, Solubility, Polymerization, Colloid and Surface Properties, and Biochemistry, John Wiley & Sons: New York (1979).

W. Stöber et al. described controlled growth of monodisperse silica spheres in the micron size range in J. Colloid and Interface Sci., 26:62-69 (1968). Stöber et al. describe a system of chemical reactions which permit the controlled growth of spherical silica particles of uniform size by means of hydrolysis of alkyl silicates and subsequent condensation of silicic acid in alcoholic solutions. Ammonia is used as a morphological catalyst. Particle sizes obtained in suspension range from less than 0.05 μm to 2 μm in diameter.

To prepare a nonporous bead, the nonporous particle can be coated with a polymer or reacted and endcapped so that substantially all surface substrate groups of the nonporous particle are blocked with a non-polar hydrocarbon or substituted hydrocarbon group. This can be accomplished by any of several methods described in U.S. Pat. No. 6,056,877. Care should be taken during the preparation of the beads to ensure that the surface of the beads has minimum silanol or metal oxide exposure and that the surface remains nonporous. Nonporous silica core beads can be obtained from Micra Scientific (Northbrook, Ill.) and from Chemie Uetikkon (Lausanne, Switzerland).

Another example of a suitable stationary support is a wide pore silica-based alkylated support as described in U.S. Pat. No. 6,379,889.

In another embodiment of the present invention, the IP-RP-HPLC separation medium can be in the form of a polymeric monolith, e.g., a rod-like monolithic column. A monolith is a polymer separation media, formed inside a column, having a unitary structure with through pores or interstitial spaces that allow eluting solvent and analyte to pass through and which provide the non-polar separation surface, as described in U.S. Pat. No. 6,066,258 and U.S. patent application Ser. No. 09/562,069. Monolithic columns, including capillary columns, can also be used, such as disclosed in U.S. Pat. No. 6,238,565; U.S. patent application Ser. No. 09/562,069 filed May 1, 2000; the PCT application WO00/15778; and by Huber et al (Anal. Chem. 71:3730-3739 (1999)). The interstitial separation surfaces can be porous, but are preferably nonporous. The separation principles involved parallel those encountered with bead-packed columns. As with beads, pores traversing the monolith must be compatible with and permeable to DNA. In a preferred embodiment, the rod is substantially free of contamination capable of reacting with DNA and interfering with its separation, e.g., multivalent cations.

A molded polymeric monolith rod that can be used in practicing the present invention can be prepared, for example, by bulk free radical polymerization within the confines of a chromatographic column. The base polymer of the rod can be produced from a variety of polymerizable monomers. For example, the monolithic rod can be made from polymers, including mono- and di-vinyl substituted aromatic compounds such as styrene, substituted styrenes, alpha-substituted styrenes and divinylbenzene; acrylates and methacrylates; polyolefins such as polypropylene and polyethylene; polyesters; polyurethanes; polyamides; polycarbonates; and substituted polymers including fluorosubstituted ethylenes commonly known under the trademark TEFLON. The base polymer can also be mixtures of polymers, non-limiting examples of which include poly(glycidyl methacrylate-co-ethylene dimethacrylate), poly(styrene-divinylbenzene) and poly(ethylvinylbenzene-divinylbenzene. The rod can be unsubstituted or substituted with a substituent such as a hydrocarbon alkyl or an aryl group. The alkyl group optionally has 1 to 1,000,000 carbons inclusive in a straight or branched chain, and includes straight chained, branch chained, cyclic, saturated, unsaturated nonionic functional groups of various types including aldehyde, ketone, ester, ether, alkyl groups, and the like, and the aryl groups includes as monocyclic, bicyclic, and tricyclic aromatic hydrocarbon groups including phenyl, naphthyl, and the like. In a preferred embodiment, the alkyl group has 1-24 carbons. In a more preferred embodiment, the alkyl group has 1-8 carbons. The substitution can also contain hydroxy, cyano, nitro groups, or the like which are considered to be non-polar, reverse phase functional groups. Methods for hydrocarbon substitution are conventional and well-known in the art and are not an aspect of this invention. The preparation of polymeric monoliths is by conventional methods well known in the art as described in the following references: Wang et al.(1994) J. Chromatog. A 699:230; Petro et al. (1996) Anal. Chem. 68:315 and U.S. Pat. Nos. 5,334,310; 5,453,185 and 5,522,994. Monolith or rod columns are commercially available form Merck & Co (Darmstadt, Germany).

The separation medium can take the form of a continuous monolithic silica gel. A molded monolith can be prepared by polymerization within the confines of a chromatographic column (e.g., to form a rod) or other containment system. A monolith is preferably obtained by the hydrolysis and polycondensation of alkoxysilanes. A preferred monolith is derivatized in order to produce non-polar interstitial surfaces. Chemical modification of silica monoliths with ocatdecyl, methyl or other ligands can be carried out. An example of a preferred derivatized monolith is one which is polyfunctionally derivatized with octadecylsilyl groups. The preparation of derivatized silica monoliths can be accomplished using conventional methods well known in the art as described in the following references which are hereby incorporated in their entirety herein: U.S. Pat. No. 6,056,877, Nakanishi, et al., J. Sol-Gel Sci. Technol. 8:547 (1997); Nakanishi, et al., Bull, Chem. Soc. Jpn. 67:1327 (1994); Cabrera, et al., Trends Analytical Chem. 17:50 (1998); Jinno, et al., Chromatographia 27:288 (1989).

MIPC is characterized by the use of a separation medium that is substantially free of metal contaminants or other contaminants that can bind DNA. Preferred beads and monoliths have been produced under conditions where precautions have been taken to substantially eliminate any multivalent cation contaminants (e.g. Fe(III), Cr(III), or colloidal metal contaminants), including a decontamination treatment, e.g., an acid wash treatment. Only very pure, non-metal containing materials should be used in the production of the beads in order to minimize the metal content of the resulting beads.

In addition to the separation medium being substantially metal-free, to achieve optimum peak separation the separation column and all process solutions held within the column or flowing through the column are preferably substantially free of multivalent cation contaminants (e.g. Fe(III), Cr(III), and colloidal metal contaminants). As described in U.S. Pat. Nos. 5,772,889, 5,997,742 and 6,017,457, this can be achieved by supplying and feeding solutions that enter the separation column with components that have process solution-contacting surfaces made of material which does not release multivalent cations into the process solutions held within or flowing through the column, in order to protect the column from multivalent cation contamination. The process solution-contacting surfaces of the system components are preferably material selected from the group consisting of titanium, coated stainless steel, passivated stainless steel, and organic polymer. Metals found in stainless steel, for example, do not harm the separation, unless they are in an oxidized or colloidal partially oxidized state. For example, 316 stainless steel frits are acceptable in column hardware, but surface oxidized stainless steel frits harm the DNA separation.

For additional protection, multivalent cations in mobile phase solutions and sample solutions entering the column can be removed by contacting these solutions with multivalent cation capture resin before the solutions enter the column to protect the separation medium from multivalent cation contamination. The multivalent capture resin is preferably cation exchange resin and/or chelating resin.

Trace levels of multivalent cations anywhere in the solvent flow path can cause a significant deterioration in the resolution of the separation after multiple uses of an IP-RP-HPLC column. This can result in increased cost caused by the need to purchase replacement columns and increased downtime. Therefore, effective measures are preferably taken to prevent multivalent metal cation contamination of the separation system components, including separation media and mobile phase contacting. These measures include, but are not limited to, washing protocols to remove traces of multivalent cations from the separation media and installation of guard cartridges containing cation capture resins, in line between the mobile phase reservoir and the IP-RP-HPLC column. These, and similar measures, taken to prevent system contamination with multivalent cations have resulted in extended column life and reduced analysis downtime.

There are two places where multivalent-cation-binding agents, e.g., chelators, are used in MIPC separations. In one embodiment, these binding agents can be incorporated into a solid through which the mobile phase passes. Contaminants are trapped before they reach places within the system that can harm the separation. In these cases, the functional group is attached to a solid matrix or resin (e.g., a flow-through cartridge, usually an organic polymer, but sometimes silica or other material). The capacity of the matrix is preferably about 2 mequiv./g. An example of a suitable chelating resin is available under the trademark CHELEX 100 (Dow Chemical Co.) containing an iminodiacetate functional group.

In another embodiment, the multivalent cation-binding agent can be added to the mobile phase. The binding functional group is incorporated into an organic chemical structure. The preferred multivalent cation-binding agent fulfills three requirements. First, it is soluble in the mobile phase. Second, the complex with the metal is soluble in the mobile phase. Multivalent cation-binding agents such as EDTA fulfill this requirement because both the chelator and the multivalent cation-binding agent-metal complex contain charges, which makes them both water-soluble. Also, neither precipitate when acetonitrile, for example, is added. The solubility in aqueous mobile phase can be enhanced by attaching covalently bound ionic functionality, such as, sulfate, carboxylate, or hydroxy. A preferred multivalent cation-binding agent can be easily removed from the column by washing with water, organic solvent or mobile phase. Third, the binding agent must not interfere with the chromatographic process.

The multivalent cation-binding agent can be a coordination compound. Examples of preferred coordination compounds include water soluble chelating agents and crown ethers. Non-limiting examples of multivalent cation-binding agents which can be used in the present invention include acetylacetone, alizarin, aluminon, chloranilic acid, kojic acid, morin, rhodizonic acid, thionalide, thiourea, α-furildioxime, nioxime, salicylaldoxime, dimethylglyoxime, α-furildioxime, cupferron, α-nitroso-β-naphthol, nitroso-R-salt, diphenylthiocarbazone, diphenylcarbazone, eriochrome black T, PAN, SPADNS, glyoxal-bis(2-hydroxyanil), murexide, α-benzoinoxime, mandelic acid, anthranilic acid, ethylenediamine, glycine, triaminotriethylamine, thionalide, triethylenetetramine, EDTA, metalphthalein, arsonic acids, α,α′-bipyridine, 4-hydroxybenzothiazole, 8-hydroxyquinaldine, 8-hydroxyquinoline, 1,10-phenanthroline, picolinic acid, quinaldic acid, α,α′,α″-terpyridyl, 9-methyl-2,3,7-trihydroxy-6-fluorone, pyrocatechol, salicylic acid, tiron, 4-chloro-1,2-dimercaptobenzene, dithiol, mercaptobenzothiazole, rubeanic acid, oxalic acid, sodium diethyldithiocarbarbamate, and zinc dibenzyldithiocarbamate. These and other examples are described by Perrin in Organic Complexing Reagents: Structure, Behavior, and Application to Inorganic Analysis, Robert E. Krieger Publishing Co. (1964). In the present invention, a preferred multivalent cation-binding agent is EDTA.

To achieve high-resolution chromatographic separations of polynucleotides, it is generally necessary to tightly pack the chromatographic column with the solid phase polymer beads. Any known method of packing the column with a column packing material can be used in the present invention to obtain adequate high-resolution separations. Typically, a slurry of the polymer beads is prepared using a solvent having a density equal to or less than the density of the polymer beads. The column is then filled with the polymer bead slurry and vibrated or agitated to improve the packing density of the polymer beads in the column. Mechanical vibration or sonication is typically used to improve packing density.

For example, to pack a 50×4.6 mm I.D. column, 2.0 grams of beads can be suspended in 10 mL of methanol with the aid of sonication. The suspension is then packed into the column using 50 mL of methanol at 8,000 psi of pressure. This improves the density of the packed bed.

There are several types of counterions suitable for use with IP-RP-HPLC. These include a mono-, di-, or trialkylamine that can be protonated to form a positive counter charge or a quaternary alkyl substituted amine that already contains a positive counter charge. The alkyl substitutions may be uniform (for example, triethylammonium acetate or tetrapropylammonium acetate) or mixed (for example, propyldiethylammonium acetate). The size of the alkyl group may be small (methyl) or large (up to 30 carbons) especially if only one of the substituted alkyl groups is large and the others are small. For example octyldimethylammonium acetate is a suitable counterion agent. Preferred counterion agents are those containing alkyl groups from the ethyl, propyl or butyl size range.

Without intending to be bound by any particular theory, it is believed the alkyl group functions by imparting a nonpolar character to the DNA through an ion pairing process so that the DNA can interact with the nonpolar surface of the separation media. The requirements for the degree of nonpolarity of the counterion-DNA pair depends on the polarity of the separation media, the solvent conditions required for separation, the particular size and type of fragment being separated. For example, if the polarity of the separation media is increased, then the polarity of the counterion agent may have to be adjusted to match the polarity of the surface and increase interaction of the counterion-DNA pair. In general, as the size and hydrophobicity of the alkyl group is increased, the separation is less influenced by DNA sequence and base composition, but rather is based predominately on DNA sequence length.

In some cases, it may be desired to increase the range of concentration of organic solvent used to perform the separation. For example, increasing the alkyl chain length on the counterion agent will increase the nonpolarity of the counterion-DNA pair resulting in the need to either increase the concentration of the mobile phase organic solvent, or increase the strength of the organic solvent type, e.g., acetonitrile is about two times more effective than methanol for eluting DNA. There is a positive correlation between concentration of the organic solvent required to elute a fragment from the column and the length of the fragment. However, at high organic solvent concentrations, the polynucleotide can precipitate. To avoid precipitation, a more non-polar organic solvent and/or a smaller counterion alkyl group can be used. The alkyl group on the counterion agent can also be substituted with halides, nitro groups, or the like to modulate polarity.

The mobile phase preferably contains a counterion agent. Typical counterion agents include trialkylammonium salts of organic or inorganic acids, such as lower alkyl primary, secondary, and lower tertiary amines, lower trialkyammonium salts and lower quaternary alkyalmmonium salts. Lower alkyl refers to an alkyl radical of one to six carbon atoms, as exemplified by methyl, ethyl, n-butyl, i-butyl, t-butyl, isoamyl, n-pentyl, and isopentyl. Examples of counterion agents include octylammonium acetate, octadimethylammonium acetate, decylammonium acetate, octadecylammonium acetate, pyridiniumammonium acetate, cyclohexylammonium acetate, diethylammonium acetate, propylethylammonium acetate, propyldiethylammonium acetate, butylethylammonium acetate, methylhexylammonium acetate, tetramethylammonium acetate, tetraethylammonium acetate, tetrapropylammonium acetate, tetrabutylammonium acetate, dimethydiethylammonium acetate, triethylammonium acetate, tripropylammonium acetate, tributylammonium acetate, tetrapropylammonium acetate, and tetrabutylammonium acetate. Although the anion in the above examples is acetate, other anions may also be used, including carbonate, phosphate, sulfate, nitrate, propionate, formate, chloride, and bromide, or any combination of cation and anion. These and other agents are described by Gjerde, et al. in Ion Chromatography, 2^{nd} Ed., Dr. Alfred Hüthig Verlag Heidelberg (1987). In a particularly preferred embodiment of the invention the counterion is tetrabutylammonium bromide (TBAB) is preferred, although other quaternary ammonium reagents such as tetrapropyl or tetrabutyl ammonium salts can be used. Alternatively, a trialkylammonium salt, e.g., triethylammonium acetate (TEAA) can be used. The pH of the mobile phase is preferably within the range of about pH 5 to about pH 9, and optimally within the range of about pH 6 to about pH 7.5.

In another aspect, the instant invention involves compositions useful in the analysis of mtDNA by DHPLC. These compositions include PCR primers which are selected to generate separate amplicons that overlap and that span the entire mitochondrial genome. The length of the overlap is at least 50 base pairs, and preferably at least 100 base pairs, and more preferably at least 500 base pairs. A non-limiting example of such a composition includes primer pairs selected from a group consisting of forward primers and their respective reverse primers (Table 1), wherein the forward primers consist of

SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35,

and wherein the reverse primers consist of

SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, and SEQ ID NO:36.

Another composition includes amplicons prepared from PCR primers which are selected to generate separate amplicons that overlap and that span the entire mitochondrial genome as described herein. The length of the overlap is at least 50 base pairs, and preferably at least 100 base pairs, and more preferably at least 500 base pairs. One example is the amplicons prepared by using the primer pairs as indicated in Table 1. The DNA template for preparing these amplicons can be obtained from a biological sample. Non-limiting examples of such a sample include tissue or cells from a patient afflicted by a mitochondrial disease; cells or tissue from a patient who is not afflicted by a mitochondrial disease; or cells from a human lymphoblast cell culture line, such as CHR, 9947A or K562. Another example of a useful DNA template for preparing such amplicons is SRM2392.

Still another composition of the invention includes the product of restriction enzyme digestion of the separate amplicons prepared from PCR primers which are selected to generate separate amplicons that overlap and that span the entire mitochondrial genome as described herein. The restriction enzymes can include one or more of the following: AluI, DdeI, HaeIII, MboI, MspI, BfaI, NlaIII, HpaII, TaqI, HinfI, HphI, SfaNI, and DpnII. Preferably, all of the restriction enzymes require about the same reaction temperature. A preferred composition is obtained from the use of one or more of the following restriction enzymes: MboI, HaeIII, DdeI, MspI, and AluI. The size of the fragments produced after the digestion is preferably in the range of about 70 to about 700 base pairs. Yet another composition of the instant invention concerns the product of a denaturation and re-annealing procedure carried out on the products restriction enzyme cleavage of the separate amplicons as described.

In another aspect, the invention concerns kits for use in determining the presence of a mutation in mtDNA by DHPLC. A kit of the invention can include one or more of the following:

a plurality of pre-selected primer pairs for amplifying said entire genome by the polymerase chain reaction, wherein the pre-selected primer pairs are selected such that amplicons obtained using the primer pairs comprise overlapping segments of the entire human mitochondrial genome, each primer contained in a separate container,

the primer pairs can be selected from a group consisting of forward primers and their respective reverse primers, wherein the forward primers consist of

SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35,

and wherein the reverse primers consist of

SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, and SEQ ID NO:36;

one or more pre-selected restriction enzymes for cleaving amplicons obtained using these primer pairs, wherein the enzymes are selected such that, for each of these primer pairs, the DNA products after the amplifying and the cleaving are between about 50 base pairs and about 700 base pairs in length, and each of said pre-selected restriction enzymes is contained in a separate container;

in separate containers, restriction enzymes selected from the group consisting of one or more or the following: AluI, DdeI, HaeIII, MboI, MspI, BfaI, NIaIII, HpaII, TaqI, HinfI, HphI, SfaNI, and DpnII. Preferably all of the restriction enzymes require about the same reaction temperature. A preferred kit contains, in separate container, restriction enzymes selected from the group of one or more of MboI, HaeIII, DdeI, MspI, and AluI;

a reverse phase column containing separation beads, or a monolithic column, for separating double stranded DNA by denaturing high performance liquid chromatography;

a chromatography system for performing denaturing high performance liquid chromatography;

one or more DNA polymerases, each in a separate container. One or more of the DNA polymerase is preferably a proof reading polymerase. Non-limiting examples of the proof reading polymerase include Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT™, DEEPVENT™, PFUTurbo™, AmpliTaq®, AccuType™ and active mutants, variants and derivatives thereof. Preferred polymerase for use in the kit includes at least one of Pho (Optimase polymerase), Taq, Pfu or mixtures thereof;

in a separate container, control DNA, such as SRM 2393, corresponding to the entire mitochondrial genome;

in a separate container, DNA obtained from muscle of an individual who is not afflicted with mitochondrial disease;

in a separate container, a control biological sample;

in a separate container, cells from human lymphoblast cell culture line CHR;

in a separate container, cells from human lymphoblast cell culture line 9947A;

in a separate container, cells from human lymphoblast cell culture line K562;

a set of pre-selected primer pairs for amplifying the entire human genome by the polymerase chain reaction, wherein the pre-selected primer pairs are selected such that amplicons obtained using these primers comprise overlapping segments of the entire mitochondrial genome, each primer contained in a separate container. The kit can further include one or more pre-selected restriction enzymes for cleaving amplicons obtained using these primer pairs, wherein the enzymes are selected such that, for each of the primer pairs, the DNA products after the amplifying and cleaving are between about 50 base pairs and about 700 base pairs in length;

instructional material.

The various aspects of the invention as described herein provide methods, compositions, and kits to scan for genetic alterations in the human mitochondrial genome. Commercially available cell lines as described herein can be used to validate the accuracy of this procedure to detect polymorphic changes dispersed throughout the 16.5 kb sequence. This approach can be used to detect the presence of both inherited or somatic mtDNA base changes. Identification and characterization of these base changes is an important first step in the challenging task of assigning functional consequences to alterations in the mitochondrial genome. The instant invention will contribute to the growing understanding of the complexities of mitochondrial pathology and can be used for a variety of different applications involving mtDNA.

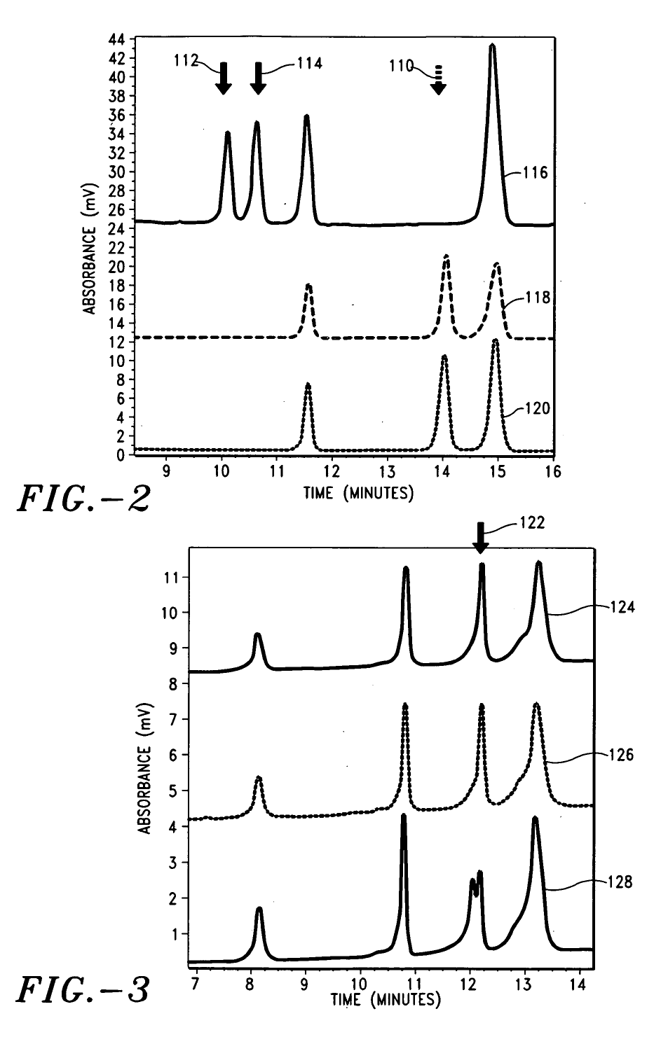

One advantage of the method of the invention is that once a heteroduplex peak is detected, the corresponding region of the mitochondrial genome can be immediately located and sequenced using conventional techniques. For example, a heteroduplex peak was detected in the mixed MT set # 9 CHR and MT set #9 9947A sample (FIG. 3; elution time 12.2 minutes). Table 2 indicates that this 233 bp peak corresponds to mitochondrial base pairs 6029-6261. The mitochondrial genome scan can be used to rapidly identify regions in mtDNA that contain putative base changes, so only a limited number of sequencing reactions are needed to confirm the DHPLC results. In the practice of the method, careful examination of the elution order of the peaks at different temperatures is preferred to ensure the proper peak is identified for further analysis, since each fragment has different melting characteristics. The elution profiles can be complex for some fragments (FIGS. 6-9), but the CHR and 9947A positive control fragments provide a well-characterized reference to guide the identification of specific peaks, and thus the location in the mitochondrial genome for further study.

The Examples herein illustrate specific embodiments of the methods, compositions, and kits of the invention wherein the Applicants have developed a protocol to efficiently screen the 16.5 kb mitochondrial genome for mutations and polymorphisms by DHPLC. The mitochondrial genome was amplified in 18 overlapping sets and 14 out of 18 of these amplicons were digested with restriction enzymes that generated fragments between 100-600 bp. Restriction enzymes were selected that cleave the PCR products into suitable fragments for DHPLC analysis at 37° C., with consideration for the total number of enzymes required and the cost per unit of enzyme.

In the Examples, CHR and 9947A DNA samples were extracted from commercially available human lymphoblast cell culture lines. Sequence comparison of these two mtDNA molecules revealed several polymorphic base changes dispersed throughout the 16.5 kb genome. Each cell line analyzed individually represent a homoplasmic mitochondrial DNA sample, while a mixture of the two in equal proportions produces a sample that is 50% heteroplasmic for each base change. The mixed CHR and 9947A mtDNAs provide a standard to validate the feasibility and sensitivity of this approach to detect base changes in the mitochondrial genome. Twenty-one of the total 62 fragments analyzed had at least a single base change present in the mixed sample, and this method was able to detect heteroduplex peaks in all 21 positive control fragments. Thus, the CHR and 9947A mixed sample is preferably routinely included as a positive control for the inventive mitochondrial genome scanning method to detect unknown base changes in samples under investigation.

Samples suspected of harboring homoplasmic mtDNA alterations/mutations can be mixed with normal template prior to DHPLC analysis. An example of a suitable normal template is DNA derived from tissue from a non-affected individual. Other examples of normal template include DNA from non-affected tissue from the same patient who is afflicted with a mitochondrial disease, DNA from CHR cells or 9947A cells, and SRM2392.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All patent applications, patents, and literature references cited in this specification are hereby incorporated by reference in their entirety. In case of conflict or inconsistency, the present description, including definitions, will control. Unless mentioned otherwise, the techniques employed or contemplated herein are standard methodologies well known to one of ordinary skill in the art. The materials, methods and examples are illustrative only and not limiting.

All numerical ranges in this specification are intended to be inclusive of their upper and lower limits. All concentrations expressed in percentage are volume/volume.

Other features of the invention will become apparent in the course of the following descriptions of exemplary embodiments which are given for illustration of the invention and are not intended to be limiting thereof.

Procedures described in the past tense in the Examples below have been carried out in the laboratory. Procedures described in the present tense have not yet been carried out in the laboratory, and are constructively reduced to practice with the filing of this application.

### EXAMPLE 1

### Amplification of Mitochondrial DNA

Primer sets were designed to amplify the entire human mitochondrial genome (base pair numbering consistent with MITOMAP version) in eighteen overlapping segments. The primer sequences, the mitochondrial regions amplified, and the size of the polymerase chain reaction (PCR) products are listed in Table 1. The mitochondrial fragments were amplified using the Expand™ Long Template PCR System (Roche Molecular Biochemicals) in 1× PCR buffer 1 with 1.75 mM MgCl₂, 350 μM each dNTP (Applied Biosystems, Foster City, Calif.), 300 nM forward primer, 300 nM reverse primer, 1 U Expand™ Taq polymerase mix, and 50-100 ng of genomic DNA according to the manufacturer's instructions. The components were denatured at 93° C. for 2 minutes, followed by 30 cycles of 93° C. for 10 seconds; 58° C. for 30 seconds; 68° C. for 1.5 minutes, with a final extension step of 68° C. for 7 minutes. Table 1 shows the PCR primers used in the whole genome scan.

TABLE 1

| | | | | | | |
|---|---|---|---|---|---|---|
| | | SEQ | | EQ | | |
| | | ID | | ID | MT Region | Size |
| MT Set | Forward Primer (5′ to 3′) | NO: | Reverse Primer (5′ to 3′) | NO: | Amplified | (bp) |

| | | | | | | |
|---|---|---|---|---|---|---|
| MT #1 | ctc cac cat tag cac cca | 1 | gag gat ggt ggt caa ggg acc | 2 | 15974-16409 bp | 436 |
| | aag c | | | | | |
| MT #2 | tac agt caa atc cct tct | 3 | tcc agc gtc tcg caa tgc tat c | 4 | 16341-102 bp | 331 |
| | cgt cc | | | | | |
| MT #3 | ctc acg gga gct ctc cat | 5 | att agt agt atg gga gtg gga gg | 6 | 29-480 bp | 452 |
| | gca t | | | | | |
| MT #4 | acc cta aca cca gcc taa | 7 | ttg tct ggt agt aag gtg gag tg | 8 | 368-1713 bp | 1346 |
| | cca g | | | | | |
| MT #5 | aac tta act tga ccg ctc | 9 | agg ttg ggt tct gct ccg agg | 10 | 1650-2841 bp | 1192 |
| | tga gc | | | | | |
| MT #6 | ctc act gtc aac cca aca | 11 | tgt gtt gtg ata agg gtg gag ag | 12 | 2415-3811 bp | 1397 |
| | cag g | | | | | |
| MT #7 | ccc tac ggg cta cta | 13 | ccc gat agc tta ttt agc tga cc | 14 | 3429-4428 bp | 1000 |
| | caa ccc | | | | | |
| MT #8 | act tcc tac cac tca ccc | 15 | gga gat agg tag gag tag cgt g | 16 | 4180-5488 bp | 1309 |
| | tag c | | | | | |
| MT #9 | cct acg cct aat cta ctc | 17 | ccc taa gat aga gga gac acc tg | 18 | 5347-6382 bp | 1036 |
| | cac c | | | | | |
| MT #10 | ctg gag cct ccg tag acc | 19 | ggc ata cag gac tag gaa gca g | 20 | 6318-7707 bp | 1390 |
| | taa c | | | | | |
| MT #11 | tat cac ctt tca tga tca | 21 | gtc cga gga ggt tag ttg tgg c | 22 | 7644-8784 bp | 1141 |
| | cgc cc | | | | | |
| MT #12 | aac cga cta atc acc acc | 23 | gga tta tcc cgt atc gaa ggc c | 24 | 8643-9458 bbp | 816 |
| | caa ca | | | | | |
| MT #13 | aag cac ata cca agg cca | 25 | gtg gag tcc gta aag agg tat c | 26 | 9397-11397 bp | 2001 |
| | cca c | | | | | |
| MT #14 | ctc ctg agc caa caa ctt | 27 | gga ttg ctt gaa tgg ctg ctg tg | 28 | 11322-12852 bp | 1531 |
| | aat atg | | | | | |
| MT #15 | ctg ttc atc ggc tga gag | 29 | agt tga ctt gaa gtg gag aag gc | 30 | 12753-13264 bp | 512 |
| | ggc | | | | | |
| MT #16 | ctt agg cgc tat cac cac | 31 | taa gcc ttc tcc tat tta tgg gg | 32 | 13172-14610 bp | 1439 |
| | tct g | | | | | |
| MT #17 | cca tgc ctc agg ata ctc | 33 | cgg aga att gtg tag gcg aat ag | 34 | 14427-15590 bp | 1164 |
| | ctc a | | | | | |
| MT #18 | aaa gac gcc ctc ggc tta | 35 | agc gag gag agt agc act ctt g | 36 | 15424-16451 bp | 1028 |
| | ctt c | | | | | |

### EXAMPLE 2

### Restriction Enzyme Digestion

Restriction endonucleases (New England Biolabs, Beverly, Mass.) were selected that cleave the mtDNA PCR products into fragments in the range of about 100 to about 600 bp. The restriction enzymes, recommended 10× buffers, fragment sizes, and the corresponding mtDNA regions are listed in Table 2. PCR products amplified in mitochondrial sets 1, 2, 3, and 15 do not require a restriction enzyme digestion, and were set aside until the next step in the procedure. Mitochondrial sets 1-3 span the hypervariable region and were analyzed by DHPLC without a restriction enzyme digestion step because this region is highly polymorphic. Each restriction enzyme digestion was performed in a final volume of 100 μl with 88.5 μl of mtDNA PCR product, 10 μl of the recommended 10× restriction enzyme buffer, and 1.5 μl of the appropriate enzyme(s). The reactions were incubated at 37° C. for a minimum of 2 hours, and 9 μl was analyzed on the Transgenomic WAVE® DNA Fragment Analysis System at 50° C. to check for complete digestion of the PCR products. A 10 μl aliquot of the pUC18 HaeIII digest (Transgenomic) was run as a size standard.

The WAVE® system had a DNASep® column (50×4.6 mm ID) with a stationary phase consisting of 2 μm nonporous alkylated poly(styrene-divinylbenzene) particles, a UV detector set at 260 nm, and a 96-well autosampler.

The mobile phase consisted of Solvent A: 0.1M triethylammonium acetate (Transgenomic) and Solvent B: 0.1M triethylammonium acetate, 25% (v/v) acetonitrile (Transgenomic). The gradient for elution of the restriction enzyme fragments was:

| Step | Time | % A | % B |
|---|---|---|---|
| Loading | 0.0 | 65 | 35 |
| Start Gradient | 1.0 | 60 | 40 |
| Stop Gradient | 17.0 | 28 | 72 |
| Start Clean | 17.1 | 60 | 40 |
| Stop Clean | 17.2 | 60 | 40 |
| Start Equilibrate | 17.3 | 60 | 40 |
| Stop Equilibrate | 17.4 | 60 | 40 |

(The experimental results included herein were obtained using the gradient listed above. Further analysis indicated the analysis time could be reduced using the adjusted gradient of 45-67% B from 0.5 to 11.5 minutes. This gradient has the same slope as the original gradient, so the resolution of the peaks is not altered.)

Table 2 shows the result of restriction enzyme digestion of mitochondrial DNA amplicons.

TABLE 2

| Mitochondrial | Restriction | 10X NEB | Fragment | Mitochondrial |
|---|---|---|---|---|
| Set | Enzyme | Buffer | Sizes | Region (bp) |
| MT #1 | — | — | 436 | 15975-16410 |
| MT #2 | — | — | 331 | 16342-102 |
| MT #3 | — | — | 452 | 29-480 |
| MT #4 | MboI | NEB-3 | 211 | 740-950 |
| | | | 276 | 951-1226 |
| | | | 372 | 368-739 |
| | | | 487 | 1227-1713 |
| MT #5 | HaeIII | NEB-2 | 273 | 2569-2841 |
| | | | 394 | 2175-2568 |
| | | | 525 | 1650-2174 |
| MT #6 | DdeI | NEB-3 | 125 | 3067-3192 |
| | | | 210 | 2857-3066 |
| | | | 279 | 3535-3812 |
| | | | 342 | 3193-3534 |
| | | | 442 | 2415-2856 |
| MT #7 | HaeIII | NEB-2 | 109 | 3851-3959 |
| | | | 178 | 3430-3608 |
| | | | 242 | 3609-3850 |
| | | | 471 | 3960-4429 |
| MT #8 | MspI | NEB-2 | 135 | 4712-4846 |
| | | | 248 | 5243-5489 |
| | | | 396 | 4847-5242 |
| | | | 530 | 4181-4711 |
| MT #9 | HaeIII | NEB-2 | 123 | 6262-6383 |
| | | | 190 | 5839-6028 |
| | | | 233 | 6029-6261 |
| | | | 490 | 5348-5838 |
| MT #10 | MspI | NEB-2 | 117 | 6572-6688 |
| | | | 162 | 6689-6850 |
| | | | 252 | 6319-6571 |
| | | | 354 | 6850-7204 |
| | | | 505 | 7205-7708 |
| MT #11 | HaeIII | NEB-2 | 141 | 8252-8392 |
| | | | 181 | 8393-8573 |
| | | | 213 | 8574-8785 |
| | | | 242 | 7645-7887 |
| | | | 364 | 7888-8251 |
| MT #12 | DdeI | NEB-3 | 188 | 9273-9459 |
| | | | 238 | 8644-8882 |
| | | | 390 | 8883-9272 |
| MT #13 | AluI | NEB-2 | 247 | 9398-9645 |
| | | | 312 | 10600-10911 |
| | | | 366 | 10234-10599 |
| | | | 440 | 10912-11398 |
| | | | 588 | 9646-10233 |
| MT #14 | HaeIII + MspI | NEB-2 | 178 | 12124-12301 |
| | | | 365 | 11323-11688 |
| | | | 435 | 11689-12123 |
| | | | 553 | 12302-12853 |
| MT #15 | — | — | 512 | 12754-13265 |
| MT #16 | AluI + DdeI | NEB-2 | 129 | 14306-14434 |
| | | | 174 | 14435-14611 |
| | | | 289 | 14017-14305 |
| | | | 381 | 13173-13554 |
| | | | 462 | 13555-14016 |
| MT #17 | MboI | NEB-3 | 191 | 14869-15059 |
| | | | 236 | 15357-15591 |
| | | | 297 | 15060-15356 |
| | | | 440 | 14428-14868 |
| MT #18 | AluI | NEB-2 | 218 | 15778-15995 |
| | | | 352 | 15425-15777 |
| | | | 458 | 15996-16452 |

### EXAMPLE 3

### Heteroduplex Detection by Denaturing High Performance Liquid Chromatography

Digested mtDNA PCR products and MT sets 1, 2, 3, and 15 were analyzed by DHPLC using the same gradient conditions shown in Example 2 for the restriction enzyme fragments. Products from each cell line were analyzed individually, and an equal amount of product from each cell line was mixed together and analyzed by DHPLC to detect the presence of single nucleotide polymorphisms (SNPs). The samples were heated to 95° C. for 5 minutes and cooled slowly to room temperature (−0.1° C./sec) to allow the formation of heteroduplex molecules. The column temperatures for each fragment were predicted by Wavemaker software 4.0 (Transgenomic) with minor adjustments. An injection volume of 12 μl was sufficient for each sample at each temperature, but this may need to be increased if the PCR product yield is low.

Table 3 shows the screening temperatures used for heteroduplex detection in the hybridized mitochondrial DNA fragments.

TABLE 3

| | Fragment | Oven Temperature |
|---|---|---|
| MT Set | Size | (° C.) |
| MT #1 | 436 | 58 |
| MT #2 | 331 | 60 |
| MT #3 | 452 | 57, 59 |
| MT #4 | 211 | 57-59 |
| | 276 | 57, 58 |
| | 372 | 57 |
| | 487 | 57, 58 |
| MT #5 | 273 | 59 |
| | 394 | 56 |
| | 525 | 56 |
| MT #6 | 125 | 57 |
| | 210 | 58 |
| | 279 | 59 |
| | 342 | 59 |
| | 442 | 59 |
| MT #7 | 109 | 60 |
| | 178 | 58, 59 |
| | 242 | 57, 58 |
| | 471 | 57 |
| MT #8 | 135 | 56 |
| | 248 | 57 |
| | 396 | 56, 57 |
| | 530 | 55 |
| MT #9 | 123 | 59 |
| | 190 | 59 |
| | 233 | 58 |
| | 490 | 58 |
| MT #10 | 117 | 55 |
| | 162 | 57, 58 |
| | 252 | 57, 58 |
| | 354 | 57-59 |
| | 505 | 56 |
| MT #11 | 141 | 56 |
| | 181 | 56 |
| | 213 | 56 |
| | 242 | 56 |
| | 364 | 59 |
| MT #12 | 188 | 59 |
| | 238 | 57 |
| | 390 | 57 |
| MT #13 | 247 | 57, 58 |
| | 312 | 54, 55 |
| | 366 | 54, 55 |
| | 440 | 57 |
| | 588 | 54, 55 |
| MT #14 | 178 | 56 |
| | 365 | 56 |
| | 435 | 58 |
| | 553 | 54 |
| MT #15 | 512 | 59 |
| MT #16 | 129 | 59 |
| | 174 | 55, 56 |
| | 289 | 55-57 |
| | 381 | 57 |
| | 462 | 55-57 |
| MT #17 | 191 | 59 |
| | 236 | 57, 58 |
| | 297 | 57-59 |
| | 440 | 56, 57 |
| MT #18 | 218 | 55 |
| | 352 | 57 |
| | 458 | 57 |

### EXAMPLE 4

### Restriction Enzyme Analysis

Mitochondrial PCR fragments amplified from CHR and 9947A cell lines were digested with the appropriate restriction enzymes and run on the WAVE System at 50° C. DNA fragments were separated on the basis of size at this non-denaturing temperature. This step is critical to ensure the expected number of peaks is produced and that no partial digestion products are present. All PCR products and the restriction enzyme fragments from the CHR and 9947A cell lines had the predicted number of peaks with elution times consistent with their expected sizes. FIG. 2 shows the products of an AluI restriction enzyme digestion of MT set 18 amplified from K562, CHR, and 9947A cell lines. This example illustrates the potential problems that can result when a restriction fragment length polymorphism (RFLP) is present in one of the samples. The expected peaks with elution times of 11.5, 14, and 15 minutes were present in the CHR and 9947A cell lines. However, the peak with the elution time of 14 minutes is absent in the K562 cell line (shown as an arrow 110 with a dotted line in FIG. 2) and two additional peaks are present with elution times of 10 and 10.5 minutes (shown as arrows 112, 114 with solid lines). These results indicate that an additional recognition site for AluI is present in the K562 mitochondrial DNA sequence, therefore this cell line should not be used as a control for MT seT 18.

In FIG. 2, genomic DNA from different cell lines was amplified with MT Set # 18 primers and digested with the restriction enzyme AluI at 37° C. for 2 hours. The products were analyzed by DHPLC at a column temperature of 50° C. Cell lines CHR (profile 116) and 9947A (profile 118) show the expected AluI fragments of 218 bp, 353 bp, and 457 bp. The restriction enzyme pattern of the K562 (profile 120) cell line is missing the 353 bp fragment (arrow with dashed line), but 2 smaller bands ˜165 bp and 180 bp are present (arrows with solid line). This pattern indicates that the K562 mitochondrial DNA sequence contains an additional AluI site within the 353 bp fragment. This example demonstrates the importance of analyzing all mitochondrial fragments by DHPLC at 50° C. to check that the predicted number of bands with the expected retention times are generated to avoid misinterpretation of the elution profiles at partially denaturing conditions.

### EXAMPLE 5

### Heteroduplex Detection by DHPLC

An equal amount of PCR product or restriction enzyme digested material from the CHR and 9947A cell lines was mixed, heated to 95° C. to denature the DNA strands, and cooled slowly to allow the formation of heteroduplex molecules. Comparison of the peak areas on the WAVE® System at a column temperature of 50° C. was a convenient method to determine the relative concentration of each sample. In most cases, the yield of the PCR products was similar, so those samples were mixed in equal volumes. Each cell line was analyzed separately and run under the same conditions as the mixed samples as a control. WAVEMAKER® 4.0 software was used to predict the column temperature to detect the presence of heteroduplex molecules in each fragment. Comparison of the published mitochondrial DNA base pair changes between the CHR and 9947A cell lines (Levin et al. 1999) resulted in 32 single nucleotide polymorphisms that map to 21 separate PCR or restricition enzyme fragments, as shown in Table 4 which shows the mitochondrial DNA sequence comparison of CHR and 9947A cell lines.

TABLE 4

| | Location in | Restriction Fragment | | |
|---|---|---|---|---|
| MT Set | MITOMAP | Size (bp) | CHR | 9947A |
| 2 | 73 | 331 | G | A |
| | 93 | 452 | A | G |
| | 204 | 452 | C | T |
| 3 | 207 | 452 | A | G |
| | 214 | 452 | A | G |
| | 3092 | 452 | — | ins(C) |
| 4 | 709 | 372 | A | G |
| 5 | 1719 | 525 | A | G |
| | 2706 | 273 | G | A |
| 7 | 4135 | 471 | T | C |
| 8 | 5186 | 396 | G | A |
| 9 | 6221 | 233 | C | T |
| 10 | 6371 | 252 | T | C |
| | 6791 | 162 | G | A |
| | 7028 | 354 | T | C |
| | 7645 | 505 | T | C |
| 11 | 7861 | 242 | T | C |
| | 8448 | 181 | T | C |
| | 8503 | 181 | C | T |
| 12 | 9315 | 188 | T | C |
| 14 | 11719 | 435 | A | G |
| | 11878 | 435 | C | T |
| | 12612 | 553 | G | A |
| | 12705 | 553 | T | C |
| 16 | 13572 | 462 | T | C |
| | 13708 | 462 | A | G |
| | 13759 | 462 | G | A |
| | 13966 | 462 | G | A |
| | 14470 | 174 | C | T |
| 17 | 14767 | 440 | T | C |
| 18 | 16183 | 458 | C | A |
| | 16189 | 458 | C | T |

DHPLC was able to detect the SNPs in 21/21 (100%) of the fragments in the CHR and 9947A mixed samples. In most cases, the SNP was easily detected because the heteroduplex molecules eluted earlier than the homoduplex molecules and generated a new peak in the elution profile compared to the CHR and 9947A individual control samples. FIG. 3 shows an example of a SNP in the MT set 9 CHR and 9947A mixed sample. Genomic DNA from CHR and 9947A cell lines was amplified with MT Set #9 primers and digested with the restriction enzyme HaeIII. The individual CHR and 9947A digested PCR products and a 50:50 mixture of the two products were denatured at 95° C. for 5 minutes and cooled slowly to room temperature. The size of the original PCR product is 1036 bp, and digestion with HaeIII resulted in fragments of 122 bp, 190 bp, 233 bp, and 491 bp. Sequence analysis of CHR and 9947A mitochondrial DNA detected a single base change between these two cell lines (6221C/T) that maps within the 233 bp fragment eluting at 12 minutes (indicated by an arrow 122 in FIG. 3). The CHR and 9947A mixed sample 128 clearly shows the formation of a heteroduplex peak at a column temperature of 58° C. compared to the single 233 bp peak in the individual samples. This example demonstrates the sensitivity of DHPLC to detect this SNP, and interpretation of the result is straightforward based on heteroduplex detection by peak shape.

In FIG. 3, genomic DNA from CHR and 9947A cell lines was amplified with MT Set #9 primers and digested with the restriction enzyme HaeIII at 37° C. for 2 hours. PCR products were first analyzed by DHPLC at 50° C. (not shown). The individual CHR and 9947A digested PCR products and a 50:50 mixture of the two products were denatured at 95° C. for 5 minutes and cooled slowly to room temperature. DHPLC analysis was performed at a column temperature of 58° C. on the ULG. The size of the original PCR product is 1036 bp, and digestion with HaeIII results in fragments of 122 bp+190 bp+233 bp+491 bp. Sequence analysis of CHR and 9947A mitochondrial DNA detected a single base change between these two cell lines (6221C/T) which maps within the 233 bp fragment (indicated by arrow 122). The elution profile 128 of the CHR and 9947A mixed sample clearly shows the formation of a heteroduplex peak compared to the single 233 bp peak in the individual samples CHR (profile 124) and 9947A (profile 126). This example demonstrates the sensitivity of the mitochondrial genome scan to detect the single nucleotide polymorphism (SNP) between these two samples. Interpretation of the results is straightforward and based on heteroduplex detection by peak shape.

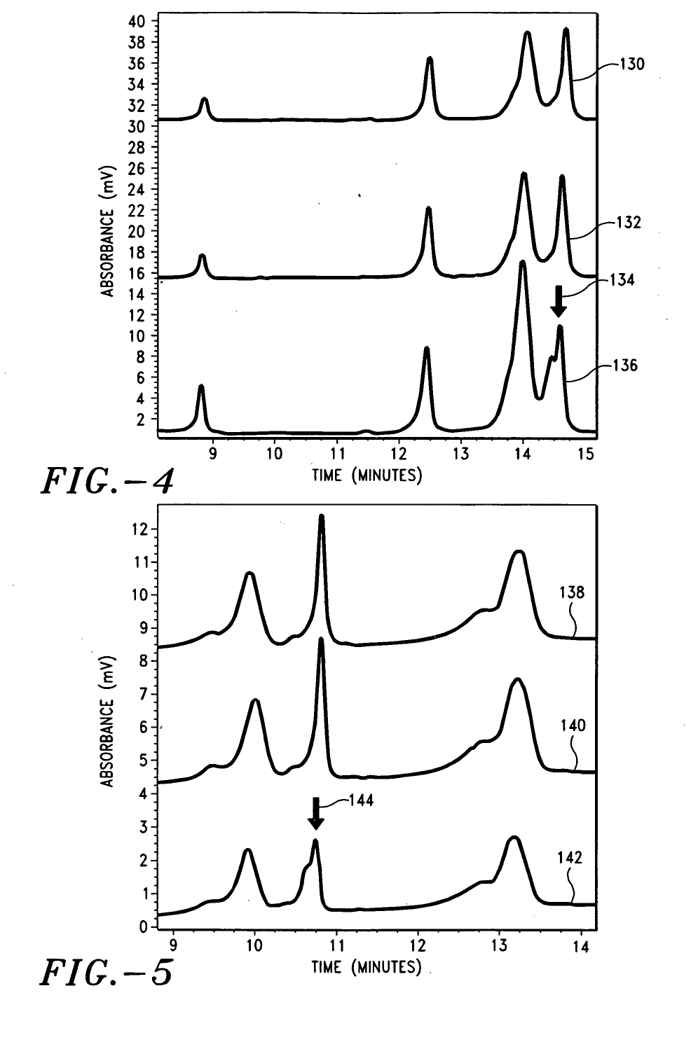

Since the multiple fragments analyzed in the mitochondrial sets are derived from a restriction enzyme digestion, the relative ratio of the peak heights within a sample can be used to detect the presence of SNPs by DHPLC. FIGS. 4 and 5 show two examples in which the peak height was used in combination with the peak shape to detect SNPs in the mixed samples. The individual CHR (profiles 130 and 138) and 9947A (profiles 132 and 140) digested PCR products and a 50:50 mixture of the two products (profiles 136 and 142) were denatured at 95° C. for 5 minutes and cooled slowly to room temperature. DHPLC analysis was performed at a column temperature of 56° C. for MT Set #8 (FIG. 4) and 59° C. for MT Set #12 (FIG. 5). Sequence analysis of the mitochondrial DNA detected base changes between these two cell lines (5186G/A in the 396 bp fragment of MT set 8; 9315T/C in the 187 bp fragment of MT set 12). Comparison of the peak heights in FIG. 4 reveals the mV intensities of the peaks eluting at 14 and 14.6 are similar in the CHR (profile 130) and 9947A (profile 132) cell lines. However, the relative ratio of the peak heights in the CHR and 9947A mixed sample (profile 136) are different from the individual samples. The peak that elutes at 14.6 minutes has approximately half the mV intensity as the peak that elutes at 14 minutes. These examples demonstrate that although heteroduplex peaks can be identified in the CHR and 9947A mixed samples containing these SNPs, interpretation of the results is most sensitive when both peak shape and peak height (mV intensity) are evaluated.

The data in FIGS. 4 and 5 suggested that there were base changes between the two cell lines in MT set 8 and MT set 12. This was confirmed in sequence analysis of CHR and 9947A mitochondrial DNA which detected base changes between these two cell lines (5186G/A-396 bp fragment MT set 8; 9315T/C-187 bp fragment MT set 12). These examples demonstrate that although heteroduplex peaks can be identified in the CHR and 9947A mixed samples containing the SNPs (indicated at arrows 134 and 144), interpretation of the results is most sensitive when both peak shape and peak height (mV intensity) are considered.

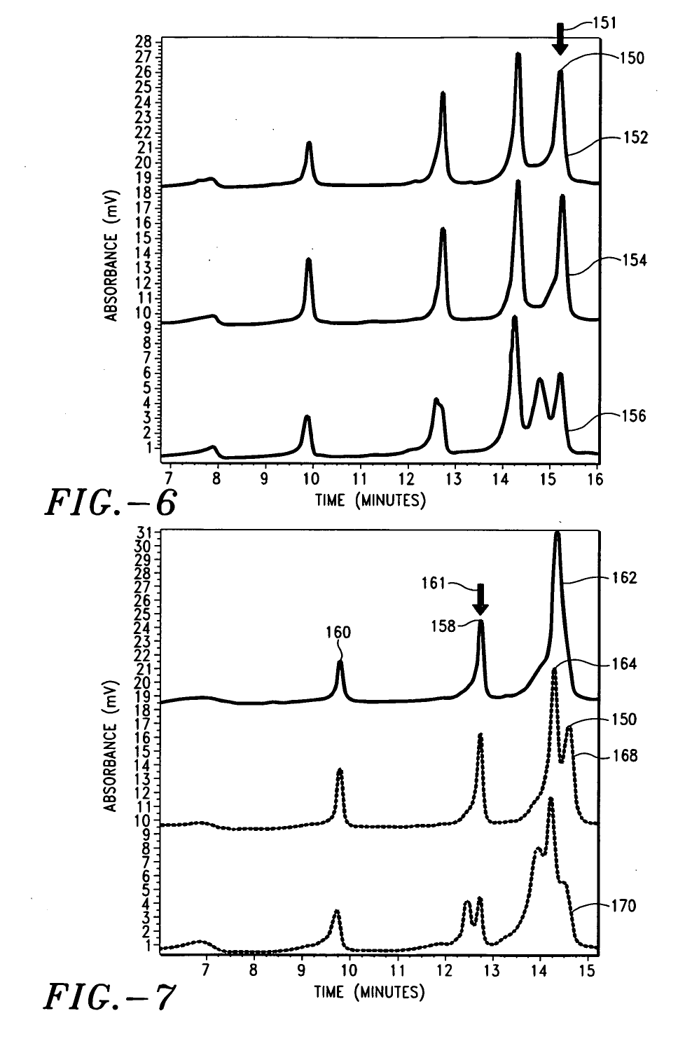

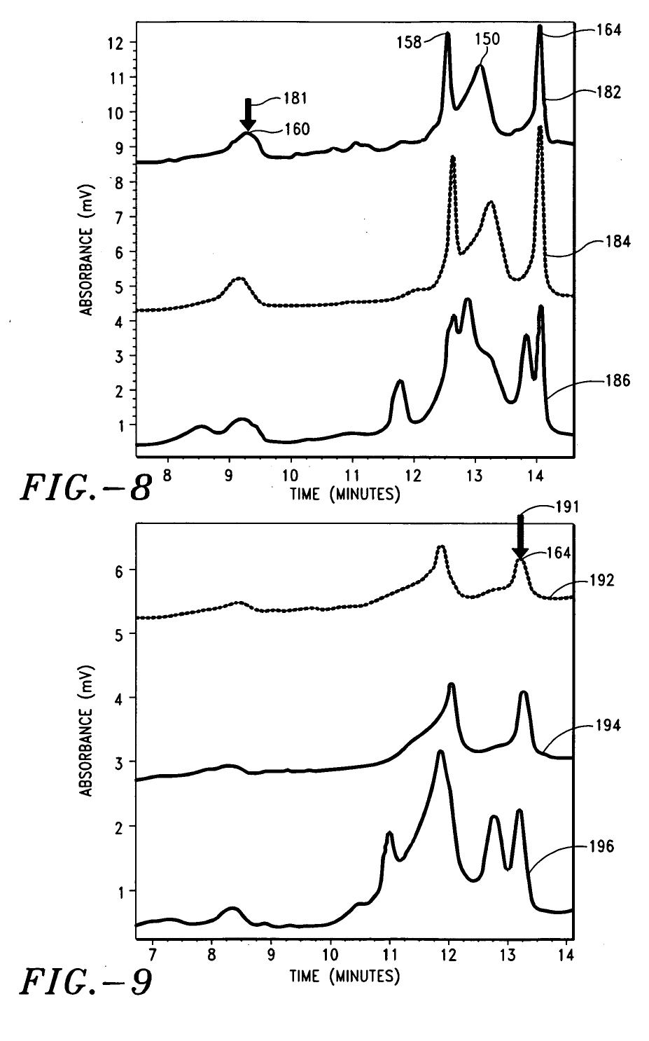

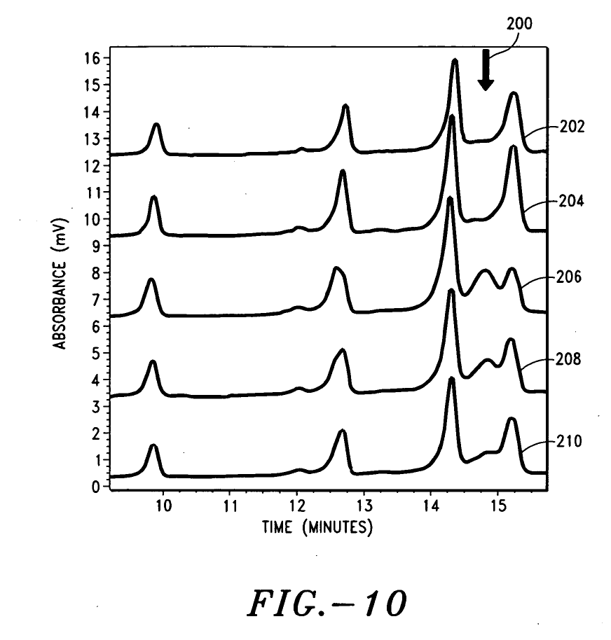

FIGS. 6-9 show the chromatograms of MT set 10 at column temperatures 56° C. (FIG. 6), 57° C. (FIG. 7), 58° C. (FIG. 8) and 59° C. (FIG. 9). Heteroduplex peaks can be clearly identified in each CHR and 9947A mixed sample containing a SNP, but interpretation of the results is more complex. The size of the MT set 10 PCR product is 1390 bp, and digestion with MspI results in fragments of 117 bp, 162 bp, 253 bp, 354 bp, and 504 bp. Sequence analysis of CHR and 9947A mitochondrial DNA detected base changes between these two cell lines (6371T/C-253 bp fragment or peak 158; 6791G/A-162 bp fragment or peak 160; 7028T/C-354 bp fragment or peak 164; 7645T/C-504 bp fragment or peak 150). The arrow (151,161, 181, 191) in each chromatogram indicates the optimal screening temperature recommended for each fragment. A heteroduplex peak can be clearly identified in each CHR and 9947A mixed sample containing the SNP, but analysis of the elution profiles is more complex because the 504 bp fragment (peak 150) has a lower melting temperature than the 354 bp fragment (peak 164), thus peak 150 elutes before peak 164 at 58° C. and 59° C.

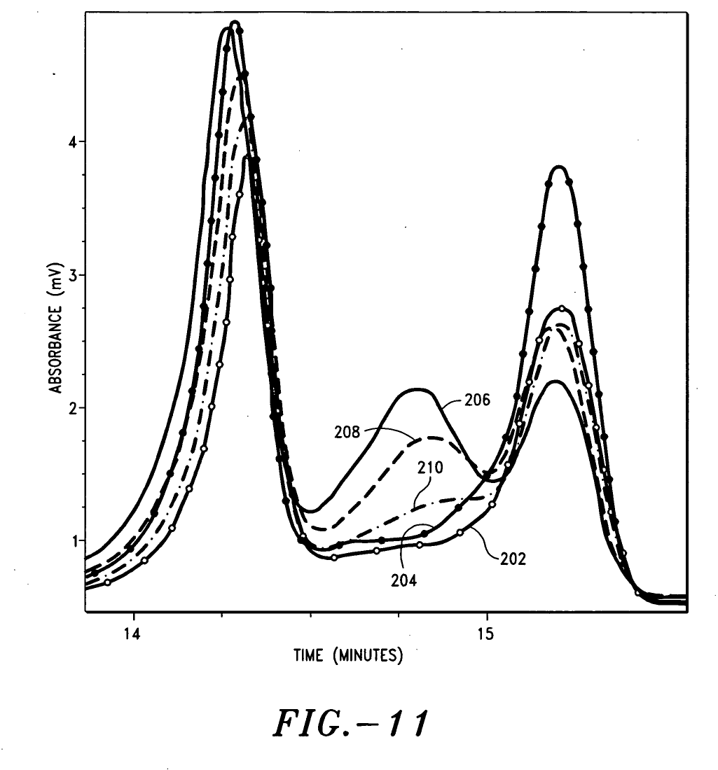

Finally, CHR and 9947A products were mixed in different ratios to determine if heteroplasmic base changes in the mitochondrial genome could be detected using this approach (FIGS. 10 and 11). Genomic DNA from CHR and 9947A cell lines was amplified with MT set 10 primers and digested with MspI at 37° C. for 2 hours. The individual CHR (profile 202) and 9947A (profile 204) digested PCR products and 50:50 (profile 206), 80:20 (profile 208), and 90:10 (profile 210) mixtures of the two products were denatured at 95° C. for 5 minutes and cooled slowly to room temperature. DHPLC analysis was performed at a column temperature of 56° C. The CHR and 9947A samples show a single peak, while a heteroduplex peak is detected in the CHR:9947A mixed samples (arrow 200). This example demonstrates the feasibility of scanning for heteroplasmic base changes in the mitochondrial genome using this method. This example demonstrates the sensitivity of heteroplasmy detection for this SNP is approximately between 10-20%.

While the foregoing has presented specific embodiments of the present invention, it is to be understood that these embodiments have been presented by way of example only. It is expected that others will perceive and practice variations which, though differing from the foregoing, do not depart from the spirit and scope of the invention as described and claimed herein.

All patent applications, patents, and literature references cited in this specification are hereby incorporated by reference in their entirety. In case of conflict or inconsistency, the present description, including definitions, will control.

The invention claimed is:

1. A method for detecting the presence of one or more mutations in the entire human mitochondrial genome, wherein said genome is present in a biological sample, the method comprising:
(a) amplifying DNA from said biological sample by polymerase chain reaction using a plurality of pre-selected primer pairs, wherein separate amplicons are generated from each primer pair, wherein said primer pairs are selected such that said amplicons comprise overlapping segments of said entire mitochondrial genome;
(b) cleaving at least one of said separate amplicons using pre-selected restriction enzymes, wherein said enzymes are selected such that, for each of said separate amplicons, the DNA products obtained after said amplifying and said cleaving are between about 50 base pairs and about 700 base pairs in length;
(c) for each of said separate amplicons, denaturing and re-annealing the separate amplicons of step (b);
(d) for each of said separate amplicons, analyzing the product of step (c) using denaturing high performance liquid chromatography, wherein the presence of said one or more mutations is confirmed if at least one heteroduplex is detected.

2. The method of claim 1, further including confirming that said cleaving in step (b) is complete.

3. The method of claim 2, wherein said confirming comprises analyzing the product of step (b) by ion-pairing reverse-phase high pressure liquid chromatography under non-denaturing conditions.

4. The method of claim 1, wherein said analyzing comprises:
applying said mixture to a stationary reverse phase support; and,
eluting the mixture with a mobile phase containing an ion-paring reagent and an organic solvent, where said eluting is carried out under conditions effective to at least partially denature heteroduplex molecules, and wherein said eluting results in the separation or partial separation of heteroduplex and homoduplex molecules.

5. The method of claim 1, wherein said restriction enzymes are selected from the group consisting of at least one of AluI, DdeI, HaeIII, MboI, MspI, BfaI, NlaIII, HpaII, TaqI, HinfI, HphI, SfaNI, DpnII, and mixtures thereof.

6. The method of claim 1, wherein said restriction enzymes are selected from the group consisting of at least one of MboI, HaeIII, DdeI, MspI, and AluI, and mixtures thereof.

7. The method of claim 1, wherein said restriction enzymes each require about the same reaction temperature for optimal activity.

8. The method of claim 1, wherein the number of separate amplicons is at least 5.

9. The method of claim 1, wherein the number of separate amplicons is at least 10.

10. The method of claim 1, wherein the number of separate amplicons is at least 20.

11. The method of claim 1, wherein the number of separate amplicons is in the range of about 1 to about 70.

12. The method of claim 1, wherein the number of separate amplicons is in the range of about 10 to about 50.

13. The method of claim 1, wherein the number of separate amplicons is in the range of about 15 to about 25.

14. The method of claim 1, wherein each of said separate amplicons has two neighboring amplicons, one at each end, which two neighboring amplicons overlap said each of said separate amplicons, wherein the length of said overlap is at least 50 base pairs.

15. The method of claim 1, wherein each separate amplicon has sequences at its ends which overlap the end sequences of its two neighboring amplicons, wherein said overlap is at least 100 base pairs.

16. The method of claim 1, wherein each separate amplicon has sequences at its ends which overlap the end sequences of its two neighboring amplicons, wherein said overlap is in the range of about 50 to about 1000 base pairs.

17. The method of claim 1, wherein each separate amplicon has sequences at its ends which overlap the end sequences of its two neighboring amplicons, wherein said overlap is in the range of about 60 to about 500 base pairs.

18. The method of claim 1, wherein each of said separate amplicons has two neighboring amplicons at each end, which two neighboring amplicons overlap said each of said separate amplicons, wherein the length of said overlap is at least 500 base pairs.

19. The method of claim 1, wherein each of said separate amplicons has two neighboring amplicons, one at each end, which two neighboring amplicons overlap said each of said separate amplicons, wherein the length of said overlap is in the range of about 50 to about 500 base pairs.

20. The method of claim 1, wherein, for each of said separate amplicons, the product of said cleaving comprises fragments differing in length by at least 20 base pairs.

21. The method of claim 1, wherein, for each of said separate amplicons, the product of said cleaving comprises fragments differing in length by at least 40 base pairs.

22. The method of claim 1, wherein, for each of said separate amplicons, the product of said cleaving comprises fragments differing in length by at least 100 base pairs.

23. The method of claim 1, wherein, for each of said separate amplicons, the product of said cleaving comprises fragments differing in length by at least 300 base pairs.

24. The method of claim 1, wherein said biological sample comprises muscle from a human patient suspected of having a mitochondrial disease.

25. The method of claim 1, wherein said biological sample comprises blood from a human patient suspected of having a mitochondrial disease.

26. The method of claim 1, wherein said biological sample comprises tissue from a human patient suspected of having a mitochondrial disease.

27. The method of claim 1, wherein said biological sample comprises human brain tissue.

28. The method of claim 1, wherein said biological sample comprises cells from human lymphoblast cell culture line 9947A.

29. The method of claim 1, wherein said biological sample comprises cells from human lymphoblast cell culture line CHR.

30. The method of claim 1, wherein said biological sample comprises cells from human lymphoblast cell culture line K562.

31. The method of claim 1, wherein said biological sample is a test sample obtained from a human patient suspected of having a mitochondrial disease, and further including subjecting a control sample to steps (a) through (d) wherein a control DHPLC elution profile is generated, and comparing said test sample DHPLC elution profile with said control DHPLC elution profile.

32. The method of claim 31, wherein said control sample comprises tissue from an individual not afflicted a mitochondrial disease.

33. The method of claim 11, wherein said control sample comprises standard reference material SRM2392.

34. The method of claim 31, wherein said control sample comprises cells from human lymphoblast cell culture line CHR.

35. The method of claim 31, wherein said control sample comprises cells from human lymphoblast cell culture line 9947A.

36. The method of claim 31, wherein said control sample comprises cells from human lymphoblast cell culture line K562.

37. The method of claim 1, wherein said DNA comprises total DNA from human lymphoblast cell culture line 9947A.

38. The method of claim 1, wherein said DNA comprises total DNA from human lymphoblast cell culture line CHR.

39. The method of claim 1, wherein said DNA comprises a mixture of total DNA from human lymphoblast cell culture line 9947A and total DNA from human lymphoblast cell culture line CHR.

40. The method of claim 1, wherein said biological sample is a test sample and wherein in step (d) a test sample DHPLC elution profile is generated, and further including subjecting a control sample to steps (a) through (d) wherein a control DHPLC elution profile is generated, and comparing said test sample DHPLC elution profile with said control DHPLC elution profile, wherein one or more differences between said test sample DHPLC elution profile and said control DHPLC elution profile is indicative of the presence of at least one heteroduplex.

41. The method of claim 40, wherein said test sample comprises tissue from a human patient suspected of having a mitochondrial disease, wherein said control sample comprises tissue from an individual not afflicted with a mitochondrial disease.

42. The method of claim 40, wherein said one or more differences comprise differences in elution peak height.

43. The method of claim 40, wherein said one or more differences comprise differences in elution peak shape.

44. The method of claim 40, wherein said one or more differences comprise differences in elution peak number.

45. The method of claim 1, further including extracting DNA from said biological sample prior to step (a).

46. The method of claim 1, wherein said primer pairs are selected from a group consisting of forward primers and their respective reverse primers, wherein said forward primers consist of
SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35,
and wherein said reverse primers consist of
SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, and SEQ ID NO:36.

47. A composition comprising the product of step (a) in claim 1.

48. A composition comprising the product of step (a) in claim 47, wherein said biological sample comprises cells from human lymphoblast cell culture line CHR.

49. A composition comprising the product of step (a) in claim 47, wherein said biological sample comprises cells from human lymphoblast cell culture line 9947A.

50. A composition comprising the product of step (b) in claim 1.

51. A composition comprising the product of step (c) in claim 1.

52. A DHPLC elution profile generated during step (d) in claim 1.

53. A DHPLC elution profile of claim 52, wherein said biological sample comprises cells from human lymphoblast cell culture line CHR.

54. A kit for detecting mutations in the entire human mitochondrial genome, said kit comprising:
a) pre-selected pairs of primers for amplifying said entire genome by the polymerase chain reaction, wherein said pre-selected pairs of primers are selected such that amplicons obtained using said primers comprise overlapping segments of said entire mitochondrial genome, each of said primers in said kit in a separate container; and,
b) one or more pre-selected restriction enzymes for cleaving amplification products obtained using said primers, wherein said enzymes are selected such that, for each of said primer pairs, the DNA products after said amplifying and said cleaving are between about 50 base pairs and about 700 base pairs in length, each of said restriction enzymes in said kit in a separate container.

55. The kit of claim 54, wherein said DNA products after said amplifying and said cleaving are between about 70 base pairs and about 500 base pairs in length.

56. The kit of claim 54, wherein said pairs of primers are selected from a group consisting of forward primers and their respective reverse primers, wherein said forward primers consist of
SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35,
and wherein said reverse primers consist of
SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, and SEQ ID NO:36.

57. The kit of claim 54, wherein said restriction enzymes are selected from the group consisting of at least one of AluI, DdeI, HaeIII, MboI, MspI, BfaI, NlaIII, HpaII, TaqI, HinfI, HphI, SfaNI, and DpnII.

58. The kit of claim 54, wherein said restriction enzymes are selected from the group consisting of at least one of MboI, HaeIII, DdeI, MspI, and AluI.

59. The kit of claim 54, wherein said restriction enzymes each require about the same reaction temperature for optimal activity.

60. The kit of claim 54, further including a reverse phase column for separating double stranded DNA by denaturing high performance liquid chromatography.

61. The kit of claim 54, further including a monolithic reverse phase column for separating double stranded DNA by denaturing high performance liquid chromatography.

62. The kit of claim 54, further including a disc having hydrophobic separation surfaces for separating double stranded DNA.

63. The kit of claim 54, further including a chromatography system for performing denaturing high performance liquid chromatography.

64. The kit of claim 54, further including at least one DNA polymerase.

65. The kit of claim 64, wherein said at least one DNA polymerase comprises a proof reading polymerase.

66. The kit of claim 65, wherein said proof reading polymerase comprises Pho polymerase.

67. The kit of claim 66, wherein said proof reading polymerase comprises Taq polymerase.

68. The kit of claim 66, wherein said proof reading polymerase comprises Pfu polymerase.

69. The kit of claim 66, wherein said proof reading polymerase comprises a mixture of Pfu and Taq polymerase.

70. The kit of claim 54, further including control DNA corresponding to said entire mitochondrial genome.

71. The kit of claim 70, wherein said control DNA is from cells of lymphoblast cell culture line CHR.

72. The kit of claim 70, wherein said control DNA is from cells of lymphoblast cell culture line 9947.

73. The kit of claim 70, wherein said control DNA is obtained from tissue of an individual who is not afflicted with mitochondrial disease.

74. The kit of claim 70, wherein said control DNA comprises standard reference material SRM 2392.

75. The kit of claim 70, wherein said control DNA is from tissue of an individual who is not afflicted with mitochondrial disease.

76. The kit of claim 54, further including a control sample.

77. The kit of claim 76, wherein said control sample comprises cells from human lymphoblast cell culture line CHR.

78. The kit of claim 76, wherein said control sample comprises cells from human lymphoblast cell culture line 9947.

79. A kit for detecting mutations in the entire human mitochondrial genome, said kit comprising:
a set of pre-selected pairs of primers for amplifying said entire genome by the polymerase chain reaction, wherein said pre-selected pairs of primers are selected such that amplicons obtained using said primers comprise overlapping segments of said entire mitochondrial genome, each of said primers in a separate container.

80. The kit of claim 79, wherein the number of separate amplicons is in the range of about 15 to about 25.

81. The kit of claim 79, wherein each of said separate amplicons has two neighboring amplicons, one at each end, which two neighboring amplicons overlap said each of said separate amplicons, wherein the length of said overlap is in the range of about 50 to about 1000 base pairs.

82. The kit of claim 79, wherein said pairs of primers are selected from a group consisting of forward primers and their respective reverse primers,
wherein said forward primers consist of
SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35,
and wherein said reverse primers consist of
SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, and SEQ ID NO:36.

83. The kit of claim 79, further including one or more pre-selected restriction enzymes for cleaving amplicons obtained using said primers, wherein said enzymes are selected such that, for each of said primer pairs, the DNA products after said amplifying and said cleaving are between about 50 base pairs and about 700 base pairs in length.

84. The kit of claim 83, wherein said restriction enzymes are selected from the group consisting of at least one of AluI, DdeI, HaeIII, MboI, MspI, BfaI, NIaIII, HpaII, TaqI, HinfI, HphI, SfaNI, and DpnII.

85. The kit of claim 83, wherein said restriction enzymes are selected from the group consisting of at least one of MboI, HaeIII, DdeI, MspI, and AluI.

86. The kit of claim 83, wherein said restriction enzymes each require about the same reaction temperature for optimal activity.

87. A method for detecting the presence of one or more mutations in the entire human mitochondrial genome, wherein said genome is present in a test biological sample, the method comprising:
(a) amplifying DNA from said test sample by polymerase chain reaction using a plurality of pre-selected primer pairs, wherein separate amplicons are generated for each primer pair, wherein said primer pairs are selected such that said amplicons comprise overlapping segments of said entire mitochondrial genome, wherein said amplicons comprise fragments that are greater than a size that is suitable for analysis by denaturing high performance liquid chromatography;
(b) cleaving at least one of said amplicons using one or more pre-selected restriction enzymes, wherein said enzymes are selected such that for each of said separate amplicons the DNA products after said amplifying and said cleaving are within a size range that is suitable for analysis by denaturing high performance liquid chromatography;
(c) denaturing and re-annealing the separate amplicons of step (b);
(d) for each of said separate amplicons, analyzing the product of step (c) using denaturing high performance liquid chromatography, wherein the presence of said one or more mutations is confirmed if at least one heteroduplex is detected.

88. The method of claim 87, further including confirming that said cleaving is complete prior to step (d).

89. The method of claim 87, wherein said size range that is suitable for analysis by denaturing high performance liquid chromatography is between about 50 base pairs and about 1000 base pairs.

90. The method of claim 87, wherein said size range that is suitable for analysis by denaturing high performance liquid chromatography is between about 70 base pairs and about 500 base pairs.

91. The method of claim 87, wherein said amplicons in step (a) comprise DNA fragments that are greater than a size than is suitable for analysis by denaturing high performance liquid chromatography comprise fragments that are at least about 1000 base pairs.

92. A method for detecting the presence of one or more mutations in the entire human mitochondrial genome, wherein said genome is present in a biological sample, the method comprising:
(a) amplifying DNA from said biological sample by polymerase chain reaction using a plurality of pre-selected primer pairs, wherein separate amplicons are generated from each primer pair, wherein said primer pairs are selected such that said amplicons comprise overlapping segments of said entire mitochondrial genome;
(b) cleaving at least one of said separate amplicons using one or more pre-selected restriction enzymes, wherein said enzymes are selected such that, for each of said separate amplicons, the DNA products obtained after said amplifying and said cleaving are between about 50 base pairs and about 700 base pairs in length;
(c) for each of said separate amplicons, denaturing and re-annealing the separate amplicons of step (b);
(d) for each of said separate amplicons, analyzing the product of step (c) using denaturing high performance liquid chromatography, wherein the presence of said one or more mutations is confirmed if at least one heteroduplex is detected.

93. A method for detecting the presence of one or more mutations in the entire human mitochondrial genome, wherein said genome is present in a biological sample, the method comprising:
(a) amplifying DNA from said biological sample by polymerase chain reaction using a plurality of pre-selected primer pairs, wherein separate amplicons are generated from each primer pair, wherein said primer pairs are selected such that said amplicons comprise overlapping segments of said entire mitochondrial genome;
(b) for each of said separate amplicons, denaturing and re-annealing the separate amplicons of step (a);
(c) cleaving at least one of said separate amplicons using pre-selected restriction enzymes, wherein said enzymes are selected such that, for each of said separate amplicons, the DNA products obtained after said amplifying and said cleaving are between about 50 base pairs and about 700 base pairs in length;
(d) for each of said separate amplicons, analyzing the product of step (c) using denaturing high performance liquid chromatography, wherein the presence of said one or more mutations is confirmed if at least one heteroduplex is detected.


SEQUENCE LISTING (36 sequences)

SEQ ID NO: 1 - DNA, 22 bp, Artificial Sequence
  Forward Primer
ctccaccattagcacccaaagc

SEQ ID NO: 2 - DNA, 21 bp, Artificial Sequence
  Reverse Primer
gaggatggtggtcaagggacc

SEQ ID NO: 3 - DNA, 23 bp, Artificial Sequence
  Forward Primer
tacagtcaaatcccttctcgtcc

SEQ ID NO: 4 - DNA, 22 bp, Artificial Sequence
  Reverse Primer
tccagcgtctcgcaatgctatc

SEQ ID NO: 5 - DNA, 22 bp, Artificial Sequence
  Forward Primer
ctcacgggagctctccatgcat

SEQ ID NO: 6 - DNA, 23 bp, Artificial Sequence
  Reverse Primer
attagtagtatgggagtgggagg

SEQ ID NO: 7 - DNA, 22 bp, Artificial Sequence
  Forward Primer
accctaacaccagcctaaccag

SEQ ID NO: 8 - DNA, 23 bp, Artificial Sequence
  Reverse Primer
ttgtctggtagtaaggtggagtg

SEQ ID NO: 9 - DNA, 23 bp, Artificial Sequence
  Forward Primer
aacttaacttgaccgctctgagc

SEQ ID NO: 10 - DNA, 21 bp, Artificial Sequence
  Reverse Primer
aggttgggttctgctccgagg

SEQ ID NO: 11 - DNA, 22 bp, Artificial Sequence
  Forward Primer
ctcactgtcaacccaacacagg

SEQ ID NO: 12 - DNA, 23 bp, Artificial Sequence
  Reverse Primer
tgtgttgtgataagggtggagag

SEQ ID NO: 13 - DNA, 21 bp, Artificial Sequence
  Forward Primer
ccctacgggctactacaaccc

SEQ ID NO: 14 - DNA, 23 bp, Artificial Sequence
  Reverse Primer
cccgatagcttatttagctgacc

SEQ ID NO: 15 - DNA, 22 bp, Artificial Sequence
  Forward Primer
acttcctaccactcaccctagc

SEQ ID NO: 16 - DNA, 22 bp, Artificial Sequence
  Reverse Primer
ggagataggtaggagtagcgtg

SEQ ID NO: 17 - DNA, 22 bp, Artificial Sequence
  Forward Primer
cctacgcctaatctactccacc

SEQ ID NO: 18 - DNA, 23 bp, Artificial Sequence
  Reverse Primer
ccctaagatagaggagacacctg

SEQ ID NO: 19 - DNA, 22 bp, Artificial Sequence
  Forward Primer
ctggagcctccgtagacctaac

SEQ ID NO: 20 - DNA, 22 bp, Artificial Sequence
  Reverse Primer
ggcatacaggactaggaagcag

SEQ ID NO: 21 - DNA, 23 bp, Artificial Sequence
  Forward Primer
tatcacctttcatgatcacgccc

SEQ ID NO: 22 - DNA, 22 bp, Artificial Sequence
  Reverse Primer
gtccgaggaggttagttgtggc

SEQ ID NO: 23 - DNA, 23 bp, Artificial Sequence
  Forward Primer
aaccgactaatcaccacccaaca

SEQ ID NO: 24 - DNA, 22 bp, Artificial Sequence
  Reverse Primer
ggattatcccgtatcgaaggcc

SEQ ID NO: 25 - DNA, 22 bp, Artificial Sequence
  Forward Primer
aagcacataccaaggccaccac

SEQ ID NO: 26 - DNA, 22 bp, Artificial Sequence
  Reverse Primer
gtggagtccgtaaagaggtatc

SEQ ID NO: 27 - DNA, 24 bp, Artificial Sequence
  Forward Primer
ctcctgagccaacaacttaatatg

SEQ ID NO: 28 - DNA, 23 bp, Artificial Sequence
  Reverse Primer
ggattgcttgaatggctgctgtg

SEQ ID NO: 29 - DNA, 21 bp, Artificial Sequence
  Forward Primer
ctgttcatcggctgagagggc

SEQ ID NO: 30 - DNA, 23 bp, Artificial Sequence
  Reverse Primer
agttgacttgaagtggagaaggc

SEQ ID NO: 31 - DNA, 22 bp, Artificial Sequence
  Forward Primer
cttaggcgctatcaccactctg

SEQ ID NO: 32 - DNA, 23 bp, Artificial Sequence
  Reverse Primer
taagccttctcctatttatgggg

SEQ ID NO: 33 - DNA, 22 bp, Artificial Sequence
  Forward Primer
ccatgcctcaggatactcctca

SEQ ID NO: 34 - DNA, 23 bp, Artificial Sequence
  Reverse Primer
cggagaattgtgtaggcgaatag

SEQ ID NO: 35 - DNA, 22 bp, Artificial Sequence
  Forward Primer
aaagacgccctcggcttacttc

SEQ ID NO: 36 - DNA, 22 bp, Artificial Sequence
  Reverse Primer
agcgaggagagtagcactcttg